(19) United States
(12) Patent Application Publication
(10) Pub. No.: US 2018/0201998 A1
(43) Pub. Date: Jul. 19, 2018

(54) COMPOSITIONS AND METHODS FOR DETECTION OF GENETIC DEAFNESS GENE MUTATION

(71) Applicants: CapitalBio Corporation, Beijing (CN); Tsinghua University, Beijing (CN)

(72) Inventors: Guangxin XIANG, Beijing (CN); Xuezhong LIU, Beijing (CN); Xingping CHAI, Beijing (CN); Wanli XING, Beijing (CN); Jing CHENG, Beijing (CN)

(73) Assignees: CapitalBio Corporation, Beijing (CN); Tsinghua University, Beijing (CN)

(21) Appl. No.: 15/744,719

(22) PCT Filed: Jul. 14, 2015
(86) PCT No.: PCT/CN2015/000505

§ 371 Date: Jan. 12, 2018

(51) Int. Cl. C12Q 1/6883

(52) U.S. Cl. CPC C12Q 1/6883; C12Q 2600/16; C12Q 2600/156

(57) ABSTRACT

In one aspect, a kit for detection of genetic gene mutation is provided, which is used for detecting nine deafness gene mutations of Caucasian populations, including GJB2 (c.35delG, c.167delT, c.132G>C, and c.269T>C), GJB6 (c.del309kb), SLC26A4 (c.707T>C and c.1246A>C), 12S rRNA (m.1555A>G and m.7444G>A). In another aspect, a method is provided, which method comprises labeling a target molecule with a luminophore, coupling the target molecule to a particle, and binding to a probe molecule on microarray. In some aspects, this technology, with high sensitivity, enables the detection and interpretation of molecular interactions in an efficient way.

### TECHNICAL FIELD

In some aspects, the present disclosure relates to the area of bioassays. In particular aspects, it is related to a microarray-based method for analyzing molecular interactions, e.g., multiplexed genetic analysis of nucleic acid fragments, including diagnosis of clinical samples and hearing loss-associated testing.

### BACKGROUND

In recently years, microarray technologies enable the evaluation of up to tens of thousands of molecular interactions simultaneously in a high-throughput manner. DNA microarray-based assays have been widely used, including the applications for gene expression analysis, genotyping for mutations, single nucleotide polymorphisms (SNPs), and short tandem repeats (STRs), with regard to drug discovery, disease diagnostics, and forensic purpose (Heller, Ann Rev Biomed Eng (2002) 4: 129-153; Stoughton, Ann Rev Biochem (2005) 74: 53-82; Hoheisel, Nat Rev Genet (2006) 7: 200-210). Pre-determined specific oligonucleotide probes immobilized on microarray can serve as a de-multiplexing tool to sort spatially the products from parallel reactions performed in solution (Zhu et al., Antimicrob Agents Chemother (2007) 51: 3707-3713), and even can be more general ones, i.e., the designed and optimized artificial tags or their complementary sequences employed in the universal microarray (Gerrey et al., J Mol Biol (1999) 292: 251-262; Li et al., Hum Mutat (2008) 29: 306-314). Combined with the multiplex PCR method, microarray-based assays for SNPs and gene mutations, such as deletions, insertions, and indels, thus can be carried out in routine genetic and diagnostic laboratories. However, it's still highly desirable to further improve both sensitivity and specificity of microarray-based assays, concerning with the detection of various SNPs and gene mutations, particularly in clinical settings.

### SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In some aspects, the present disclosure is directed at compositions and methods for analyzing molecular interactions, e.g., multiplex investigation of interactions between pharmaceutical compounds, and multiplex detection of genetic information using microarray-based technology combined with particles, in particular microparticles.

In one aspect, the present disclosure provides a method for detecting a target molecule using a microarray, which method comprises: a) labeling the target molecule with a luminophore; b) coupling the target molecule to a particle; c) binding the target molecule to a probe molecule immobilized on the microarray; and d) detecting the interaction between the target molecule and the probe molecule, wherein the target molecule is selected from the group consisting of a polynucleotide, a polypeptide, an antibody, a small molecule compound, a peptide and a carbohydrate.

In another aspect, the present disclosure provides method for detecting a target molecule using a microarray, which method comprises: a) labeling a double-stranded target molecule with a luminophore; b) coupling the double-stranded target molecule to a particle; c) recovering a single stranded target molecule with the luminophore coupled to the particle; d) binding the single stranded target molecule to a probe molecule immobilized on the microarray; and e) detecting the interaction between the target molecule and the probe molecule, wherein the target molecule is a polynucleotide, a polypeptide, an antibody, a peptide and a carbohydrate.

Any suitable luminophore can be used in the present disclosure. In one aspect, a target molecule is labeled with a luminophore. For a double-stranded target molecule, in one aspect, the harvested single-stranded molecule is labeled with luminophore, while the other single strand may or may not be labeled.

Any suitable particle can be used in the present methods. Each particle may be coupled with at least one target molecule. In one embodiment, the particle is a microparticle. In another embodiment, the microparticle is a paramagnetic microsphere. In some embodiments, the microparticle has a diameter from about 0.1 micrometers to about 10 micrometers. In other embodiments, the particle or microsphere is modified with a labeling or other functional moiety such as a fluorophore, a silver-staining reagent, a chemiluminescence reagent, an electrochemical reagent, or a nano-particle, a quantum dot, or a combination thereof.

The particle may be coated with a functional group. In one embodiment, the functional group may be selected from the group consisting of a chemical group, a polynucleotide, a polypeptide, an antibody, a small molecule compound, a peptide and a carbohydrate. In another embodiment, the chemical group may be aldehyde, hydroxyl, carboxyl, ester, amine, sulfo, or sulfhydryl. In yet another embodiment, the functional group may be selected from the group consisting of streptavidin, neutravidin and avidin. In still another embodiment, the polynucleotide is poly-dT or poly-dA.

The target molecule may be modified. In some embodiments, the target molecule is modified in addition to being labeled with one or more luminophores. The modification of the target molecule may be selected from the group consisting of a chemical group, a polynucleotide, a polypeptide, an antibody, a small molecule compound, a peptide and a carbohydrate. In some embodiments the chemical group may be aldehyde, hydroxyl, carboxyl, ester, amine, sulfo, or sulfhydryl. In some other embodiments, the polypeptide may be streptavidin, neutravidin, or avidin. In yet other embodiments, the polynucleotide may be poly-dT or poly-dA. In some embodiments, the target molecule is coupled to the particle through an interaction between the modification and the functional group. In some other embodiments, the interaction is a streptavidin-biotin interaction, a neutravidin-biotin interaction, an avidin-biotin interaction, or a poly-dT/poly-dA interaction.

The target polynucleotide may be double stranded or single stranded. In some embodiments, at least a portion of the single-stranded target polynucleotide is completely or substantially complementary to at least a portion of the oligonucleotide probe immobilized on the microarray. In other embodiments, the single-stranded target polynucleotide is completely complementary to the oligonucleotide probe immobilized on the microarray.

The target polynucleotide may be subject to an in vitro manipulation, which may produce single-stranded or double-stranded polynucleotide fragments. In one embodiment, physical treatment is employed including laser, ultrasonication, heat, microwave, piezoelectricity, electrophoresis, dielectrophoresis, solid phase adhesion, filtration and fluidic stress. In one embodiment, the in vitro manipulation is selected from the group consisting of enzymatic digestion, PCR amplification, reverse-transcription, reverse-transcription PCR amplification, allele-specific PCR (ASPCR), single-base extension (SBE), allele specific primer extension (ASPE), restriction enzyme digestion, strand displacement amplification (SDA), transcription mediated amplification (TMA), ligase chain reaction (LCR), nucleic acid sequence based amplification (NASBA), primer extension, rolling circle amplification (RCA), self sustained sequence replication (3 SR), the use of Q Beta replicase, nick translation, and loop-mediated isothermal amplification (LAMP).

For the double-stranded target polynucleotide, they may be denatured by any suitable method, e.g., a chemical reaction, an enzymatic reaction or physical treatment such as heating, or a combination thereof. In some embodiments, the chemical reaction uses urea, formamide, methanol, ethanol, sodium hydroxide, or a combination thereof. In some embodiments, enzymatic methods include exonuclease and Uracil-N-glycosylase. In other embodiments, the double-stranded target polynucleotide is heat denatured at an appropriate temperature from about 30° C. to about 95° C.

In one embodiment, the microarray comprises at least two probe molecules. In another embodiment, the microarray comprises multiple oligonucleotide probes. In yet another embodiment, the probe molecule is selected from the group consisting of a polynucleotide, a polypeptide, an antibody, a small molecule compound, a peptide and a carbohydrate.

In one embodiment, the single-stranded target polynucleotide obtained may comprise an artificially designed and optimized polynucleotide sequence such as a Tag sequence. In yet another embodiment, the microarray comprises a universal Tag array.

In still another embodiment, the Tag sequences are complementary or substantially complementary to the oligonucleotide probes on the universal Tag array.

The Tm difference between different Tag sequences can be set at any suitable range, e.g., equals or is less than about 5° C., e.g., about 5° C., 4.9° C., 4.8° C., 4.7° C., 4.6° C., 4.5° C., 4.4° C., 4.3° C., 4.2° C., 4.1° C., 4.0° C., 3.5° C., 3.0° C., 2.5° C., 2.0° C., 1.5° C., 1.0° C., or less. In some embodiments, the Tag sequences have no cross-hybridization among themselves. In some other embodiments, the Tag sequences have low homology to the genomic DNA of the species. In preferred embodiments, the Tag sequences have no hair-pin structures. In one embodiment, the Tag sequence is a single stranded oligonucleotide or modified analog. In another embodiment, the Tag sequence is a locked nucleic acid (LNA), a zip nucleic acid (ZNA) or a peptide nucleic acid (PNA). In yet another embodiment, the Tag sequence is introduced to the target polynucleotide during an in vitro manipulation.

The microarray can be made by any suitable methods. In some embodiments, the microarray is fabricated using a technology selected from the group consisting of printing with fine-pointed pins, photolithography using pre-made masks, photolithography using dynamic micromirror devices, ink-jet printing, microcontact printing, and electrochemistry on microelectrode arrays. Supporting material of the microarray may be selected from the group consisting of silicon, glass, plastic, hydrogel, agarose, nitrocellulose and nylon.

The probe molecule immobilized on the microarray may be selected from the group consisting of a polynucleotide, a polypeptide, an antibody, a small molecule compound, a peptide and a carbohydrate. The probe may be attached to the microarray in any suitable fashion, such as in situ synthesis, nonspecific adsorption, specific binding, nonspecific chemical ligation, or chemoselective ligation. The binding between the probe and the microarray may be a covalent bond or physical adhesion. The supporting material of the microarray may be any suitable material, e.g., silicon, glass, plastic, hydrogel, agarose, nitrocellulose and nylon. A spot on the microarray may have any suitable size. In one embodiment, a spot on the microarray ranges from about 10 micrometers to about 5000 micrometers in diameter. In another embodiment, the oligonucleotide probe is a single stranded oligonucleotide or modified analog. In yet another embodiment, the oligonucleotide probe is a LNA, a ZNA or a PNA. The binding between the target molecule and the probe molecule may be a non-covalent, reversible covalent or irreversible covalent interaction.

An external force including a magnetic force and a dielectrophoretic force may be applied to manipulate the particle or microsphere so as to enhance the efficiency and efficacy of the binding between the target molecule and the probe molecule. The result may be detected any suitable means, e.g., with a microarray scanning device for luminescence, an ordinary image-capturing device, or a naked eye. In one embodiment, the microarray scanning device employs optical detection with a fluorescent label, a chemiluminescent label or an enzyme. In another embodiment, the microarray scanning device employs electrochemical detection with an enzyme, a ferrocene label or other electroactive label. In yet another embodiment, the microarray scanning device employs label-free detection based on surface plasmon resonance, magnetic force, giant magnetoresistance or microgravimetric technique. In still another embodiment, the ordinary image-capturing device is a flatbed scanner, a camera, or a portable device. In some embodiments, the detection result is recorded by a camera with or without the assistance of a lens, a magnifier, or a microscope. In some other embodiments, the detection result is recorded by a portable device with a camera including a mobile phone and a laptop computer with or without the assistance of a lens, a magnifier, or a microscope.

In one embodiment, the target molecule is associated with a disease caused by an infectious or pathogenic agent selected from the group consisting of a fungus, a bacterium, a mycoplasma, a rickettsia, a chlamydia, a virus and a protozoa. In another embodiment, the target molecule is associated with a sexually transmitted disease, cancer, cerebrovascular disease, heart disease, respiratory disease, coronary heart disease, diabetes, hypertension, Alzheimer's disease, neurodegenerative disease, chronic obstructive pulmonary disease, autoimmune disease, cystic fibrosis, spinal muscular atrophy, beta thalassemia, phenylalanine hydroxylase deficiency, Duchenne muscular dystrophy, or hereditary hearing loss. In yet another embodiment, the target molecule is associated with hereditary hearing loss.

The present methods can be used for any suitable purposes. In one aspect, the present invention provides a method for detecting a genetic information, which method comprises: a) labeling a target molecule with a luminophore; b) coupling the target molecule to a particle; b) binding the target molecule to a probe molecule immobilized on the microarray, c) detecting the interaction between the target molecule and the probe molecule, wherein the target molecule comprises the genetic information, and the target molecule is selected from the group consisting of a polynucleotide, a polypeptide, an antibody, a small molecule compound, a peptide and a carbohydrate.

In a particular aspect, the present invention provides a method for detecting a genetic information, which method comprises: a) labeling a double-stranded target molecule with a luminophore; b) coupling the double-stranded target molecule to a particle; b) recovering a single stranded target molecule coupled to the particle; c) binding the single stranded target molecule to a probe molecule immobilized on the microarray; and d) detecting the interaction between the target molecule and the probe molecule, wherein the target molecule comprises the genetic information, and the target molecule is a poly nucleotide, a polypeptide, an antibody, a peptide and a carbohydrate.

Any suitable genetic information can be detected by the present methods. For example, the genetic information may be a mutation selected from the group consisting of a substitution, an insertion, a deletion and an indel. In one embodiment, the genetic information is a single nucleotide polymorphism (SNP). In one embodiment, the genetic information is a gene. In another embodiment, the genetic information is a genetic product including a polypeptide, an antibody, a small molecule compound, a peptide and a carbohydrate.

The genetic information associated with hereditary hearing loss may be within any suitable target gene, e.g., a target gene of GJB2 (Cx26), GJB6 (Cx30), SLC26A4 (PDS), or 12S rRNA (MTRNR1). In one embodiment, the genetic information in GJB2 is selected from the group consisting of c.35delG, c.132G>C, c.167delT, and c.269T>C. In another embodiment, the genetic information in SLC26A4 is selected from the group consisting of c.707T>C and c.1246A>C. In yet another embodiment, the genetic information in 12S rRNA are m.1555A>G and m.7444G>A.

The target polynucleotide containing or suspected of containing genetic information may be amplified before detection. For example, ASPCR may be used to amplify the genetic information. Any suitable or suitable set of primers can be used in amplifying the target polynucleotide containing or suspected of containing genetic information. In one embodiment, the set of primers for the ASPCR includes at least two allele-specific primers and one common primer. In another embodiment, the allele-specific primers and the common primer have a sequence as set forth in Table 2. In yet another embodiment, the allele-specific primers terminate at the SNP or mutation locus. In still another embodiment, the allele-specific primer further comprises an artificial mismatch to the wild-type sequence. In a further embodiment, the allele-specific primers comprise a natural nucleotide or analog thereof. In some embodiments, the allele-specific primers comprise a Tag sequence. In some other embodiments, the ASPCR uses a DNA polymerase without the 3′ to 5′ exonuclease activity.

Multiple genetic information may be detected. In one embodiment, multiplex PCR is used to amplify the genetic information. The location of an oligonucleotide probe immobilized on the microarrays may serve as a de-multiplexing tool. In some embodiments, genetic materials isolated from genetic materials isolated from tissues, cells, body fluids, hair, nail and ejaculate, including saliva sample, sputum sample, sperm sample, oocyte sample, zygote sample, lymph sample, blood sample, interstitial fluid sample, urine sample, buccal swab sample, chewing gum sample, cigarette butt sample, envelope sample, stamp sample, prenatal sample, or dried blood spot sample. In other aspects, a prenatal or neonatal sample is used for the detection.

In yet another aspect, the present disclosure provides a composition comprising a luminophore-labeled target molecule coupled to a particle and a probe molecule immobilized on a microarray that binds to the target molecule, wherein the target molecule is selected from the group consisting of a polynucleotide, a polypeptide, an antibody, a small molecule compound, a peptide and a carbohydrate.

In one embodiment, the oligonucleotide probe comprises a Tag sequence as set forth in Table 1. In another embodiment, the universal Tag array comprises at least two of the Tag sequences set forth in Table 1. In yet another embodiment, the universal Tag array comprises at least four of the Tag sequences set forth in Table 1. In yet another embodiment, the universal Tag array comprises at least eight of the Tag sequences set forth in Table 1. In still another embodiment, the universal Tag array comprises all of the Tag sequences set forth in Table 1.

Further provided herein is a primer comprising a sequence as set forth in Table 2 without the Tag sequence or biotinylated universal primer sequence at the 5′-terminus, which primer is not a full-length cDNA or a full-length genomic DNA. In one embodiment, the primer consists essentially of the sequence as set forth in Table 2 without the Tag sequence or biotinylated universal primer sequence at the 5′-terminus. In another embodiment, the primer consists of the sequence as set forth in Table 2 without the Tag sequence or biotinylated universal primer sequence at the 5′-terminus. In some embodiments, the primer comprises the sequence as set forth in Table 2.

In another aspect, the primer is labeled with a luminophore so that luminophores can be introduced to the target molecules.

In one aspect, also provided herein is a set of primers for ASPCR amplification of a genetic information comprising two allele-specific primers and a common primer which was labeled with a luminophore (TAMRA) at the “*T” as set forth in Table 2.

In a further aspect, the present invention provides a kit useful for detecting nine deafness gene mutations of Caucasian populations. The kit comprises an instructional manual. In one embodiment, the kit comprises a primer comprising a sequence as set forth in Table 2 without the Tag sequence or biotinylated universal primer sequence at the 5′-terminus, which primer is not a full-length genomic DNA. In another embodiment, the kit comprises the set of primers for ASPCR amplification of a genetic information comprising two allele-specific primers and a common primer as set forth in Table 2.

In yet a further aspect, the present disclosure provides a kit useful for detecting a molecular interaction comprising a particle, a microarray and a probe molecule immobilized on the microarray.

In one aspect, disclosed herein is a method for detecting a target molecule using a microarray, which method comprises: (a) labeling the target molecule with a luminophore; (b) coupling the target molecule to a particle; (c) binding the target molecule to a probe molecule immobilized on the microarray; (d) detecting the interaction between the target molecule and the probe molecule. In some aspects, the target molecule comprises a polynucleotide comprising a genetic information which comprises: (1) a genetic information within the target gene of GJB6 (Cx30); and/or (2) c.132G>C and/or c.269T>C within the target gene of GJB2; and/or (3) c.1246A>C within the target gene of SLC26A4; and/or (4) m.7444G>A within the target gene of 12S rRNA.

In one embodiment, the genetic information within the target gene of GJB6 (Cx30) is c.del309kb. In any of the preceding embodiments, the polynucleotide can further comprise one or more of the following genetic information: c.35delG within the target gene of GJB2; c.167delT within the target gene of GJB2; c.707T>C within the target gene of SLC26A4; and m.1555A>G within the target gene of 12S rRNA.

In any of the preceding embodiments, the particle can be a microparticle. In one aspect, the microparticle is a paramagnetic microsphere. In any of the preceding embodiments, the microparticle can have a diameter from about 0.1 micrometers to about 10 micrometers. In any of the preceding embodiments, the particle can be coated with a functional group. In one aspect, the functional group is selected from the group consisting of a chemical group, a polynucleotide, a polypeptide, an antibody, a small molecule compound, a peptide and a carbohydrate. In any of the preceding embodiments, the chemical group can be an aldehyde, hydroxyl, carboxyl, ester, amine, sulfo, or sulfhydryl group, the polypeptide can be streptavidin, neutravidin, or avidin, and the polynucleotide can be poly-dT or poly-dA. In any of the preceding embodiments, the target molecule can be modified. In some embodiments, the modification of the target molecule is selected from the group consisting of a chemical group, a polynucleotide, a polypeptide, an antibody, a small molecule compound, a peptide and a carbohydrate.

In any of the preceding embodiments, the target molecule can be coupled to the particle through an interaction between the modification of the target molecule and the functional group on the particle. In any of the preceding embodiments, each particle can be coupled with at least one target molecule. In any of the preceding embodiments, the target molecule can be associated with a disease caused by an infectious or pathogenic agent selected from the group consisting of a fungus, a bacterium, a mycoplasma, a rickettsia, a chlamydia, a virus and a protozoa. In any of the preceding embodiments, the target molecule can be associated with a sexually transmitted disease, cancer, cerebrovascular disease, heart disease, respiratory disease, coronary heart disease, diabetes, hypertension, Alzheimer's disease, neurodegenerative disease, chronic obstructive pulmonary disease, autoimmune disease, cystic fibrosis, spinal muscular atrophy, beta thalassemia, phenylalanine hydroxylase deficiency, Duchenne muscular dystrophy, or hereditary hearing loss.

In any of the preceding embodiments, the probe molecule can comprise a polynucleotide, a polypeptide, an antibody, a small molecule compound, a peptide, and/or a carbohydrate.

In any of the preceding embodiments, the microarray can comprise at least one Tag sequence comprising a polynucleotide sequence selected from the group consisting of SEQ ID NOs: 1-18. In any of the preceding embodiments, the microarray can comprise at least one Tag sequence comprising a polynucleotide sequence selected from the group consisting of SEQ ID NOs: 19-23.

In any of the preceding embodiments, the microarray can comprise at least one Tag sequence as set forth in Table 1. In any of the preceding embodiments, the microarray can comprise at least two probe molecules. In one aspect, the at least two probe molecules comprise a probe molecule comprising the polynucleotide sequence set forth in SEQ ID NO: 1, and a probe molecule comprising the polynucleotide sequence set forth in SEQ ID NO: 2.

In any of the preceding embodiments, the at least two probe molecules can comprise: a probe molecule comprising the polynucleotide sequence set forth in SEQ ID NO: 3, and a probe molecule comprising the polynucleotide sequence set forth in SEQ ID NO: 4; and/or a probe molecule comprising the polynucleotide sequence set forth in SEQ ID NO: 5, and a probe molecule comprising the polynucleotide sequence set forth in SEQ ID NO: 6; and/or a probe molecule comprising the polynucleotide sequence set forth in SEQ ID NO: 7, and a probe molecule comprising the polynucleotide sequence set forth in SEQ ID NO: 8; and/or a probe molecule comprising the polynucleotide sequence set forth in SEQ ID NO: 9, and a probe molecule comprising the polynucleotide sequence set forth in SEQ ID NO: 10; and/or a probe molecule comprising the polynucleotide sequence set forth in SEQ ID NO: 11, and a probe molecule comprising the polynucleotide sequence set forth in SEQ ID NO: 12; and/or a probe molecule comprising the polynucleotide sequence set forth in SEQ ID NO: 13, and a probe molecule comprising the polynucleotide sequence set forth in SEQ ID NO: 14; and/or a probe molecule comprising the polynucleotide sequence set forth in SEQ ID NO: 15, and a probe molecule comprising the polynucleotide sequence set forth in SEQ ID NO: 16; and/or a probe molecule comprising the polynucleotide sequence set forth in SEQ ID NO: 17, and a probe molecule comprising the polynucleotide sequence set forth in SEQ ID NO: 18.

In any of the preceding embodiments, the microarray can be fabricated using a technology selected from the group consisting of printing with a fine-pointed pin, photolithography using a pre-made mask, photolithography using a dynamic micromirror device, ink-jet printing, microcontact printing, and electrochemistry on a microelectrode array.

In any of the preceding embodiments, the supporting material of the microarray can be selected from the group consisting of silicon, glass, plastic, hydrogel, agarose, nitrocellulose and nylon.

In any of the preceding embodiments, a spot on the microarray can range from about 10 micrometers to about 5,000 micrometers in diameter, e.g., about 10 micrometers, 20 micrometers, 30 micrometers, 40 micrometers, 50 micrometers, 60 micrometers, 70 micrometers, 80 micrometers, 90 micrometers, 100 micrometers, 200 micrometers, 300 micrometers, 400 micrometers, 500 micrometers, 600 micrometers, 700 micrometers, 800 micrometers, 900 micrometers, 1,000 micrometers, 1,500 micrometers, 2,000 micrometers, 2,500 micrometers, 3,000 micrometers, 3,500 micrometers, 4,000 micrometers, 4,500 micrometers, or 5,000 micrometers in diameter.

In any of the preceding embodiments, the probe molecule can be attached to the microarray by in situ synthesis, nonspecific adsorption, specific binding, nonspecific chemical ligation, or chemoselective ligation.

In any of the preceding embodiments, the binding between the target molecule and the probe molecule can be a non-covalent, reversible covalent or irreversible covalent interaction. In one aspect, the efficiency and/or efficacy of the interaction is enhanced by an external force. In one embodiment, the external force is a magnetic force, a dielectrophoretic force, a mechanical force, or a combination thereof.

In any of the preceding embodiments, the target molecule can be subject to an in vitro manipulation. In some embodiments, the in vitro manipulation is selected from the group consisting of physical treatments including laser, ultrasonication, heat, microwave, piezoelectricity, electrophoresis, dielectrophoresis, solid phase adhesion, filtration and fluidic stress, and other treatments including enzymatic digestion, PCR amplification, reverse-transcription, reverse-transcription PCR amplification, allele-specific PCR (ASPCR), single-base extension (SBE), allele specific primer extension (ASPE), restriction enzyme digestion, strand displacement amplification (SDA), transcription mediated amplification (TMA), ligase chain reaction (LCR), nucleic acid sequence based amplification (NASBA), primer extension, rolling circle amplification (RCA), self sustained sequence replication (3 SR), the use of Q Beta replicase, nick translation, and loop-mediated isothermal amplification (LAMP).

In any of the preceding embodiments, the target molecule can comprise a double-stranded polynucleotide, and the double-stranded polynucleotide can be denatured to become single-stranded by a chemical reaction, an enzyme, heating, or a combination thereof. In one aspect, the enzyme is an exonuclease, a Uracil-N-glycosylase, or a combination thereof. In another aspect, the chemical reaction uses urea, formamide, methanol, ethanol, sodium hydroxide, or a combination thereof. In yet another aspect, the double-stranded polynucleotide is denatured at an appropriate temperature from about 30° C. to about 95° C., e.g., about 30° C., 35° C., 40° C., 45° C., 50° C., 55° C., 60° C., 65° C., 70° C., 75° C., 80° C., 85° C., 90° C., or 95° C.

In any of the preceding embodiments, the in vitro manipulation can be allele-specific PCR (ASPCR). In one aspect, the set of primers for the ASPCR comprises at least two allele-specific primers and one common primer. In some embodiments, the at least two allele-specific primers comprise: (a) a primer comprising the polynucleotide sequence set forth in SEQ ID NO: 24, and a primer comprising the polynucleotide sequence set forth in SEQ ID NO: 25; and/or (b) a primer comprising the polynucleotide sequence set forth in SEQ ID NO: 27, and a primer comprising the polynucleotide sequence set forth in SEQ ID NO: 28; and/or (c) a primer comprising the polynucleotide sequence set forth in SEQ ID NO: 30, and a primer comprising the polynucleotide sequence set forth in SEQ ID NO: 31; and/or (d) a primer comprising the polynucleotide sequence set forth in SEQ ID NO: 33, and a primer comprising the polynucleotide sequence set forth in SEQ ID NO: 34; and/or (e) a primer comprising the polynucleotide sequence set forth in SEQ ID NO: 36, and a primer comprising the polynucleotide sequence set forth in SEQ ID NO: 37; and/or (f) a primer comprising the polynucleotide sequence set forth in SEQ ID NO: 39, and a primer comprising the polynucleotide sequence set forth in SEQ ID NO: 40; and/or (g) a primer comprising the polynucleotide sequence set forth in SEQ ID NO: 42, and a primer comprising the polynucleotide sequence set forth in SEQ ID NO: 43; and/or (h) a primer comprising the polynucleotide sequence set forth in SEQ ID NO: 45, and a primer comprising the polynucleotide sequence set forth in SEQ ID NO: 46; and/or (i) a primer comprising the polynucleotide sequence set forth in SEQ ID NO: 48, and a primer comprising the polynucleotide sequence set forth in SEQ ID NO: 49; and/or (j) a pair of allele-specific primers as set forth in Table 2.

In any of the preceding embodiments, the common primer can comprise: (a) a primer comprising the polynucleotide sequence set forth in SEQ ID NO: 26; and/or (b) a primer comprising the polynucleotide sequence set forth in SEQ ID NO: 29; and/or (c) a primer comprising the polynucleotide sequence set forth in SEQ ID NO: 32; and/or (d) a primer comprising the polynucleotide sequence set forth in SEQ ID NO: 35; and/or (e) a primer comprising the polynucleotide sequence set forth in SEQ ID NO: 38; and/or (f) a primer comprising the polynucleotide sequence set forth in SEQ ID NO: 41; and/or (g) a primer comprising the polynucleotide sequence set forth in SEQ ID NO: 44; and/or (h) a primer comprising the polynucleotide sequence set forth in SEQ ID NO: 47; and/or (i) a primer comprising the polynucleotide sequence set forth in SEQ ID NO: 50; and/or (j) a common primer as set forth in Table 2.

In any of the preceding embodiments, the target molecule can be modified by a biotin, a digoxin, or a combination thereof. In any of the preceding embodiments, the target polynucleotide can be modified by a poly-dA or poly-dT. In any of the preceding embodiments, the target molecule can be coupled to the particle through a streptavidin/biotin interaction, a neutravidin/biotin interaction, an avidin/biotin interaction, or a poly-dT/dA interaction.

In any of the preceding embodiments, the luminophore can be selected from the group consisting of a fluorophores, a phosphor, and a chromophore. In one aspect, the fluorophore is a quantum dot, a protein (green fluorescent protein) or a small molecule dye. In some embodiments, the small molecule dye includes: xanthene derivatives (fluorescein, rhodamine, Oregon green, eosin, texas red, etc.), cyanine derivatives (cyanine, indocarbocyanine, oxacarbocyanine, thiacarbocyanine, merocyanine, etc.), naphthalene derivatives (dansyl and prodan derivatives), coumarin derivatives, oxadiazole derivatives (pyridyloxazole, nitrobenzoxadiazole, benzoxadiazole, etc.), pyrene derivatives (cascade blue, etc.), BODIPY (Invitrogen), oxazine derivatives (Nile red, Nile blue, cresyl violet, oxazine 170, etc.), acridine derivatives (proflavin, acridine orange, acridine yellow, etc.), arylmethine derivatives (auramine, crystal violet, malachite green, etc.), CF dye (Biotium), Alexa Fluor (Invitrogen), Atto and Tracy (Sigma), Tetrapyrrole derivatives (porphin, phtalocyanine, bilirubin, etc.), and others (cascade yellow, azure B, acridine orange, DAPI, Hoechst 33258, lucifer yellow, piroxicam, quinine and anthraqinone, squarylium, oligophenylenes, etc.). In some aspects, the chromophore includes retinal (used in the eye to detect light), various food colorings, fabric dyes (azo compounds), lycopene, β-carotene, anthocyanins, chlorophyll, hemoglobin, hemocyanin, and colorful minerals such as malachite and amethyst.

In any of the preceding embodiments, the target molecule can be directly labeled with a luminophore, or indirectly through the modification of the target molecule, which is selected from the group consisting of a chemical group, a polynucleotide, a polypeptide, an antibody, a small molecule compound, a peptide, and a carbohydrate.

In any of the preceding embodiments, the target molecule can be labeled with a luminophore directly, during the in vitro manipulation, or after the in vitro manipulation. In any of the preceding embodiments, the probe molecule can be a polynucleotide.

In any of the preceding embodiments, the target molecule can comprise a universal Tag sequence. In one aspect, the Tm difference between different Tag sequences equals or is less than about 5° C. In any of the preceding embodiments, the Tag sequences can have no cross-hybridization among themselves. In any of the preceding embodiments, the Tag sequences can have low homology to the genomic DNA of the species. In any of the preceding embodiments, the Tag sequences can have no hair-pin structures. In any of the preceding embodiments, the Tag sequence can be a single stranded oligonucleotide or modified analog. In any of the preceding embodiments, the Tag sequence can be a locked nucleic acid (LNA), a Zip nucleic acid (ZNA), or a peptide nucleic acid (PNA). In any of the preceding embodiments, the Tag sequence can be introduced to the target polynucleotide during an in vitro manipulation.

In any of the preceding embodiments, the detection can be by a microarray scanning device, an ordinary image-capturing device, or a naked eye. In one aspect, the microarray scanning device employs optical detection with a fluorescent label, a chemiluminescent label, a phosphore label, or a chromophore label. In another aspect, the microarray scanning device employs label-free detection based on surface plasmon resonance, magnetic force, giant magnetoresistance or microgravimetric technique. In yet another aspect, the ordinary image-capturing device is a flatbed scanner, a camera, or a portable device. In still another aspect, the camera is with or without the assistance of a lens, a magnifier, or a microscope. In some embodiments, the portable device is a camera on a mobile phone or a laptop computer with or without the assistance of a lens, a magnifier, or a microscope.

In another aspect, disclosed herein is a method for detecting a genetic information in a target molecule using a microarray, which method comprises: (a) labeling a target molecule with a luminophore, the target molecule comprising a polynucleotide comprising a genetic information which comprises: (1) a genetic information within the target gene of GJB6 (Cx30); and/or (2) c.132G>C and/or c.269T>C within the target gene of GJB2; and/or (3) c.1246A>C within the target gene of SLC26A4; and/or (4) m.7444G>A within the target gene of 12S rRNA; (b) coupling the target molecule to a particle; (c) binding the target molecule to a probe molecule immobilized on the microarray; (d) detecting the interaction between the target molecule and the probe molecule, thereby detecting the genetic information in the target molecule.

In one embodiment, the genetic information within the target gene of GJB6 (Cx30) is c.del309kb. In any of the preceding embodiments, the polynucleotide can further comprise one or more of the following genetic information: c.35delG within the target gene of GJB2; c.167delT within the target gene of GJB2; c.707T>C within the target gene of SLC26A4; and m.1555A>G within the target gene of 12S rRNA.

In any of the preceding embodiments, the genetic information can be a mutation selected from the group consisting of a substitution, an insertion, a deletion and an indel. In any of the preceding embodiments, the genetic information can be a single nucleotide polymorphism (SNP). In any of the preceding embodiments, the genetic information can be associated with a disease caused by an infectious or pathogenic agent selected from the group consisting of a fungus, a bacterium, a mycoplasma, a rickettsia, a chlamydia, a virus and a protozoa. In any of the preceding embodiments, the genetic information can be associated with a sexually transmitted disease, cancer, cerebrovascular disease, heart disease, respiratory disease, coronary heart disease, diabetes, hypertension, Alzheimer's disease, neurodegenerative disease, chronic obstructive pulmonary disease, autoimmune disease, cystic fibrosis, spinal muscular atrophy, beta thalassemia, phenylalanine hydroxylase deficiency, Duchenne muscular dystrophy, or hereditary hearing loss. In one aspect, the genetic information is associated with hereditary hearing loss.

In any of the preceding embodiments, ASPCR can be used to amplify the genetic information. In one aspect, the set of primers for the ASPCR includes at least two allele-specific primers and one common primer. In some embodiments, the at least two allele-specific primers comprise: (a) a primer comprising the polynucleotide sequence set forth in SEQ ID NO: 24, and a primer comprising the polynucleotide sequence set forth in SEQ ID NO: 25; and/or (b) a primer comprising the polynucleotide sequence set forth in SEQ ID NO: 27, and a primer comprising the polynucleotide sequence set forth in SEQ ID NO: 28; and/or (c) a primer comprising the polynucleotide sequence set forth in SEQ ID NO: 30, and a primer comprising the polynucleotide sequence set forth in SEQ ID NO: 31; and/or (d) a primer comprising the polynucleotide sequence set forth in SEQ ID NO: 33, and a primer comprising the polynucleotide sequence set forth in SEQ ID NO: 34; and/or (e) a primer comprising the polynucleotide sequence set forth in SEQ ID NO: 36, and a primer comprising the polynucleotide sequence set forth in SEQ ID NO: 37; and/or (f) a primer comprising the polynucleotide sequence set forth in SEQ ID NO: 39, and a primer comprising the polynucleotide sequence set forth in SEQ ID NO: 40; and/or (g) a primer comprising the polynucleotide sequence set forth in SEQ ID NO: 42, and a primer comprising the polynucleotide sequence set forth in SEQ ID NO: 43; and/or (h) a primer comprising the polynucleotide sequence set forth in SEQ ID NO: 45, and a primer comprising the polynucleotide sequence set forth in SEQ ID NO: 46; and/or (i) a primer comprising the polynucleotide sequence set forth in SEQ ID NO: 48, and a primer comprising the polynucleotide sequence set forth in SEQ ID NO: 49; and/or (j) a pair of allele-specific primers as set forth in Table 2.

In any of the preceding embodiments, the common primer can comprise: (a) a primer comprising the polynucleotide sequence set forth in SEQ ID NO: 26; and/or (b) a primer comprising the polynucleotide sequence set forth in SEQ ID NO: 29; and/or (c) a primer comprising the polynucleotide sequence set forth in SEQ ID NO: 32; and/or (d) a primer comprising the polynucleotide sequence set forth in SEQ ID NO: 35; and/or (e) a primer comprising the polynucleotide sequence set forth in SEQ ID NO: 38; and/or (f) a primer comprising the polynucleotide sequence set forth in SEQ ID NO: 41; and/or (g) a primer comprising the polynucleotide sequence set forth in SEQ ID NO: 44; and/or (h) a primer comprising the polynucleotide sequence set forth in SEQ ID NO: 47; and/or (i) a primer comprising the polynucleotide sequence set forth in SEQ ID NO: 50; and/or (j) a common primer as set forth in Table 2.

In any of the preceding embodiments, the common primer can be labeled with a luminophore as well as with biotinylation. In any of the preceding embodiments, the allele-specific primers can terminate at the SNP/mutation locus. In any of the preceding embodiments, the allele-specific primer can further comprise an artificial mismatch to the corresponding target sequence. In any of the preceding embodiments, the allele-specific primers can comprise a natural nucleotide or analog thereof. In any of the preceding embodiments, the allele-specific primers can comprise a Tag sequence.

In any of the preceding embodiments, the ASPCR can use a DNA polymerase without the 3′ to 5′ exonuclease activity. In any of the preceding embodiments, at least two genetic information can be detected. In any of the preceding embodiments, multiplex PCR can be used to amplify the genetic information. In any of the preceding embodiments, the genetic information can be detected in a sample selected from the group consisting of tissue, cell, body fluid, hair, nail, ejaculate, saliva, sputum, sperm, oocyte, zygote, lymph, blood, interstitial fluid, urine, buccal swab, chewing gum, cigarette butt, envelope, stamp, a prenatal sample, or dried blood spot.

In any of the preceding embodiments, the microarray can comprise at least one Tag sequence comprising a polynucleotide sequence selected from the group consisting of SEQ ID NOs: 1-18. In any of the preceding embodiments, the microarray can comprise at least one Tag sequence comprising a polynucleotide sequence selected from the group consisting of SEQ ID NOs: 19-23.

In any of the preceding embodiments, the microarray can comprise at least one Tag sequence as set forth in Table 1. In any of the preceding embodiments, the microarray can comprise at least two probe molecules. In some aspects, the at least two probe molecules comprise a probe molecule comprising the polynucleotide sequence set forth in SEQ ID NO: 1, and a probe molecule comprising the polynucleotide sequence set forth in SEQ ID NO: 2; and/or a probe molecule comprising the polynucleotide sequence set forth in SEQ ID NO: 3, and a probe molecule comprising the polynucleotide sequence set forth in SEQ ID NO: 4; and/or a probe molecule comprising the polynucleotide sequence set forth in SEQ ID NO: 5, and a probe molecule comprising the polynucleotide sequence set forth in SEQ ID NO: 6; and/or a probe molecule comprising the polynucleotide sequence set forth in SEQ ID NO: 7, and a probe molecule comprising the polynucleotide sequence set forth in SEQ ID NO: 8; and/or a probe molecule comprising the polynucleotide sequence set forth in SEQ ID NO: 9, and a probe molecule comprising the polynucleotide sequence set forth in SEQ ID NO: 10; and/or a probe molecule comprising the polynucleotide sequence set forth in SEQ ID NO: 11, and a probe molecule comprising the polynucleotide sequence set forth in SEQ ID NO: 12; and/or a probe molecule comprising the polynucleotide sequence set forth in SEQ ID NO: 13, and a probe molecule comprising the polynucleotide sequence set forth in SEQ ID NO: 14; and/or a probe molecule comprising the polynucleotide sequence set forth in SEQ ID NO: 15, and a probe molecule comprising the polynucleotide sequence set forth in SEQ ID NO: 16; and/or a probe molecule comprising the polynucleotide sequence set forth in SEQ ID NO: 17, and a probe molecule comprising the polynucleotide sequence set forth in SEQ ID NO: 18.

In yet another aspect, disclosed herein is a composition comprising a luminophore-labeled target molecule coupled to a particle and a probe molecule immobilized on a microarray that binds to the target molecule, and the target molecule comprises a polynucleotide comprising a genetic information which comprises: (1) a genetic information within the target gene of GJB6 (Cx30); and/or (2) c.132G>C and/or c.269T>C within the target gene of GJB2; and/or (3) c.1246A>C within the target gene of SLC26A4; and/or (4) m.7444G>A within the target gene of 12S rRNA. In one aspect, the genetic information within the target gene of GJB6 (Cx30) is c.del309kb. In any of the preceding embodiments, the polynucleotide can further comprise one or more of the following genetic information: c.35delG within the target gene of GJB2; c.167delT within the target gene of GJB2; c.707T>C within the target gene of SLC26A4; and m.1555A>G within the target gene of 12S rRNA.

In any of the preceding embodiments, the microarray can comprise at least one Tag sequence comprising a polynucleotide sequence selected from the group consisting of SEQ ID NOs: 1-18. In any of the preceding embodiments, the microarray can comprise at least one Tag sequence comprising a polynucleotide sequence selected from the group consisting of SEQ ID NOs: 19-23. In any of the preceding embodiments, the microarray can comprise at least one Tag sequence as set forth in Table 1.

In any of the preceding embodiments, the probe molecule can comprise a polynucleotide, a polypeptide, an antibody, a small molecule compound, a peptide and a carbohydrate. In any of the preceding embodiments, the particle can be a microparticle.

In one aspect, the microparticle is a paramagnetic microsphere. In any of the preceding embodiments, the microparticle can have a diameter from about 0.1 micrometers to about 10 micrometers, e.g., about 0.1 micrometers, 0.2 micrometers, 0.3 micrometers, 0.4 micrometers, 0.5 micrometers, 0.6 micrometers, 0.7 micrometers, 0.8 micrometers, 0.9 micrometers, 1 micrometer, 2 micrometers, 3 micrometers, 4 micrometers, 5 micrometers, 6 micrometer, 7 micrometers, 8 micrometers, 9 micrometers, or 10 micrometers.

In still another aspect, provided herein is an oligonucleotide probe comprising a Tag sequence as set forth in Table 1 or a polynucleotide sequence selected from the group consisting of SEQ ID NOs: 1-18.

In another aspect, disclosed herein is a universal Tag array comprising at least two of the Tag sequences as set forth in Table 1. In one embodiment, the universal Tag array comprises all of the Tag sequences set forth in Table 1.

In another aspect, disclosed herein is a primer comprising a sequence as set forth in Table 2 without the Tag sequence or biotinylated universal primer sequence at the 5′-terminus, which primer is not a full-length cDNA or a full-length genomic DNA. In one aspect, the primer consists essentially of the sequence as set forth in Table 2 without the Tag sequence or biotinylated universal primer sequence at the 5′-terminus. In another aspect, the primer consists of the sequence as set forth in Table 2 without the Tag sequence or biotinylated universal primer sequence at the 5′-terminus. In still another aspect, the primer comprises a sequence as set forth in Table 2.

In one aspect, disclosed herein is a set of primers for ASPCR amplification of a genetic information comprising two allele-specific primers and a luminophore-labeled common primer as set forth in Table 2, and the luminophore is TAMRA.

In another aspect, disclosed herein is a kit useful for detecting a genetic information comprising the universal Tag array of any of the preceding embodiments. In one aspect, the kit further comprises an instructional manual. In some embodiments, the kit can further comprise the primer of any of the preceding embodiments. In some embodiments, the kit can further comprise the set of primers of any of the preceding embodiments, for ASPCR amplification of a genetic information.

In one aspect, disclosed herein is a kit useful for detecting a molecular interaction comprising a particle, a microarray and at least one probe molecule immobilized on the microarray, and the at least one probe molecule comprises a polynucleotide sequence selected from the group consisting of SEQ ID NOs: 1-18. In one embodiment, the at least one probe molecule further comprises a polynucleotide sequence selected from the group consisting of SEQ ID NOs: 19-23. In any of the preceding embodiments, the at least one probe molecule can comprise at least one Tag sequence as set forth in Table 1. In any of the preceding embodiments, the particle can be a microparticle. In one embodiment, the microparticle is a paramagnetic microsphere. In any of the preceding embodiments, the microparticle can have a diameter from about 0.1 micrometers to about 10 micrometers, e.g., about 0.1 micrometers, 0.2 micrometers, 0.3 micrometers, 0.4 micrometers, 0.5 micrometers, 0.6 micrometers, 0.7 micrometers, 0.8 micrometers, 0.9 micrometers, 1 micrometer, 2 micrometers, 3 micrometers, 4 micrometers, 5 micrometers, 6 micrometer, 7 micrometers, 8 micrometers, 9 micrometers, or 10 micrometers. In any of the preceding embodiments, the particle can be coated with a functional group. In some embodiments, the functional group is selected from the group consisting of a chemical group, a polynucleotide, a polypeptide, an antibody, a small molecule compound, a peptide and a carbohydrate. In some embodiments, the chemical group is aldehyde, hydroxyl, carboxyl, ester, amine, sulfo, or sulfhydryl. In some embodiments, the polypeptide is streptavidin, neutravidin, or avidin. In other embodiments, the polynucleotide is poly-dT or poly-dA.

### BRIEF DESCRIPTION OF THE DRAWINGS

FIG. 1 is a layout of universal Tag array for de-multiplexing, corresponding to nine mutations related to hereditary hearing loss of Caucasian populations. QC and BC represent positive and negative controls of spotting efficiency, respectively. PC and NC represent positive and negative controls of hybridization, respectively. IC represents positive control of the PCR reaction. MC represents positive control of the microsphere surface-modified moieties binding with their target molecules.

FIG. 2 shows the results of detection limit evaluation using a novel sample with all wild-type alleles for nine selected mutations of Caucasian populations related to hereditary hearing loss, using universal Tag array-based assay integrated with microparticles or microspheres.

FIG. 3 shows the assay results with patient samples that contain mutant allele for nine mutations of Caucasian populations related to hereditary hearing loss, using universal Tag array-based assay integrated with microparticles or microspheres.

### DETAILED DESCRIPTION

In some embodiments, provide herein is a kit and method for detecting nine gene mutations about hereditary hearing loss, which combines microarray based assays with particles, through binding of luminophore-labeled target molecules to probe molecules, and finally de-multiplexing. In some embodiments, provided herein is a kit and method combining microarray-based assays with particles, through enriching luminophore-labeled target nucleic acid fragments, then coupling particles to microarray spots through target-probe hybridization, and finally de-multiplexing. In some embodiments, provided herein is a kit and method combining microarray-based assays with particles, through enriching luminophore-labeled double-stranded nucleic acid fragments, harvesting single-stranded nucleic acid fragments, then coupling particles to microarray spots through target-probe hybridization, and finally de-multiplexing. Besides ensuring the high sensitivity and specificity, the results displayed with luminescence can be examined with appropriate devices. In one embodiment, provided herein is a kit to detect nine mutations related to hereditary hearing loss of Caucasian populations.

A detailed description of one or more embodiments of the claimed subject matter is provided below along with accompanying figures that illustrate the principles of the claimed subject matter. The claimed subject matter is described in connection with such embodiments, but is not limited to any particular embodiment. It is to be understood that the claimed subject matter may be embodied in various forms, and encompasses numerous alternatives, modifications and equivalents. Therefore, specific details disclosed herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications, including patent documents, scientific articles and databases, referred to in this application are incorporated by reference in their entireties for all purposes to the same extent as if each individual publication were individually incorporated by reference. If a definition set forth herein is contrary to or otherwise inconsistent with a definition set forth in the patents, patent applications, published applications or other publications that are herein incorporated by reference, the definition set forth herein prevails over the definition that is incorporated herein by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y.; D. Weir & C. Blackwell, eds., Handbook of Experimental Immunology (1996), Wiley-Blackwell, all of which are herein incorporated in their entireties by reference for all purposes.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

### A. Definitions

As used herein, the singular forms “a”, “an”, and “the” include plural references unless indicated otherwise. For example, “a” dimer includes one or more dimers.

The term “molecules” as used herein can include polynucleotides, polypeptides, antibodies, small molecule compounds, peptides, and carbohydrates.

The term “particle” or “microparticle” as used herein can include small particles, generally from about 0.01 micrometers to about 1000 micrometers. In some embodiments, a “particle” or “microparticle” includes an inherent property (e.g., magnetization, fluorescence and the like) allowing identification of each particle or microparticle as belonging to a specific group. The term “microsphere” is meant to refer to a particle, preferably spherical and usually within the range of from about 0.01 micrometers to about 1000 micrometers. In some embodiment, a microsphere may consist of one or more identifying Tags (e.g., magnetization, fluorescence and the like) formed together with a polymer, glass, or other matrix, coating or the like. The term “magnetic microsphere” is meant to refer to a particle within the range of from about 0.01 micrometers to about 1000 micrometers including one or more magnetic domains with a polymer, glass, or other matrix, coating or the like. Neither the term “microsphere” or “magnetic microsphere” is meant to exclude shapes other than spherical, and such terms are meant to include other shapes such as globular, flat and the like.

The term “microarray” as used herein can include polynucleotide, polypeptide and chemical microarrays. Specific polynucleotides, polypeptides, antibodies, small molecule compounds, peptides, and carbohydrates can now be immobilized on solid surfaces to form microarrays.

The inventive technology combines microarray-based assays with particles, through binding of target molecules (e.g., luminophore-labeled target molecules) to probe molecules, and finally demultiplexing. The term “binding” is an attractive interaction between two molecules which results in a stable association in which the molecules are in close proximity to each other. Molecular binding can be classified into the following types: non-covalent, reversible covalent and irreversible covalent. Molecules that can participate in molecular binding include polypeptides, polynucleotides, carbohydrates, lipids, and small organic molecules such as pharmaceutical compounds. Polypeptides that form stable complexes with other molecules are often referred to as receptors while their binding partners are called ligands. Polynucleotides can also form stable complex with themselves or others, for example, DNA-protein complex, DNA-DNA complex, DNA-RNA complex.

The term “polypeptide” is used herein to refer to proteins, fragments of proteins, and peptides, whether isolated from natural sources, produced by recombinant techniques, or chemically synthesized. A polypeptide may have one or more modifications, such as a post-translational modification (e.g., glycosylation, etc.) or any other modification (e.g., pegylation, etc.). The polypeptide may contain one or more non-naturally-occurring amino acids (e.g., such as an amino acid with a side chain modification). Polypeptides of the disclosure typically comprise at least about 10 amino acids.

The terms “polynucleotide,” “oligonucleotide,” “nucleic acid” and “nucleic acid molecule” are used interchangeably herein to refer to a polymeric form of nucleotides of any length (such as DNA and 12s rRNA), and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acid (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles.

Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

“Nucleic acid probe” and “probe” are used interchangeably and refer to a structure comprising a polynucleotide, as defined above, that contains a nucleic acid sequence that can bind to a corresponding target. The polynucleotide regions of probes may be composed of DNA, and/or RNA, and/or synthetic nucleotide analogs.

As used herein, “complementary or matched” means that two nucleic acid sequences have at least 50% sequence identity. Preferably, the two nucleic acid sequences have at least 60%, 70%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. In another aspect, high stringency conditions are provided by hybridization in 37.5% formamide, 7.5×Denhardt's solution, 9×SSC, 0.2% SDS at 55° C., followed by washing in 0.0.03×SSC at 42° C. Low stringency hybridization refers to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa Nucleic Acids Res. 12:203 (1984).

The terms “homologous”, “substantially homologous”, and “substantial homology” as used herein denote a sequence of amino acids having at least 50%, 60%, 70%, 80% or 90% identity wherein one sequence is compared to a reference sequence of amino acids.

The percentage of sequence identity or homology is calculated by comparing one to another when aligned to corresponding portions of the reference sequence.

“Multiplexing” or “multiplex assay” herein refers to an assay or other analytical method in which the presence of multiple polynucleotide target sequences can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime).

It is understood that aspects and embodiments of the disclosure described herein include “consisting” and/or “consisting essentially of” aspects and embodiments.

Other objects, advantages and features of the present disclosure will become apparent from the following specification taken in conjunction with the accompanying drawings.

### B. Luminophore

A luminophore is an atom or atomic grouping in a chemical compound that manifests luminescence. There exist organic and inorganic luminophores. It should be stressed that the correct, textbook terminology is luminophore, not lumophore, although the latter term has been frequently but erroneously used in the chemical literature.

Luminophores can be divided into two subcategories: fluorophores and phosphors. The difference between luminophores belonging to these two subcategories is derived from the nature of the excited state responsible for the emission of photons. Some luminophores, however, cannot be classified as being exclusively fluorophores or phosphors and exist in the gray area in between. Such cases include transition metal complexes (such as ruthenium tris-2,2′-bipyridine) whose luminescence comes from an excited (nominally triplet) metal-to-ligand charge transfer (MLCT) state, but which is not a true triplet-state in the strict sense of the definition. Most luminophores consist of conjugated pi systems or transition metal complexes. There exist purely inorganic luminophores, such as zinc sulfide doped with rare earth metal ions, rare earth metal oxysulfides doped with other rare earth metal ions, yttrium oxide doped with rare earth metal ions, zinc orthosilicate doped with manganese ions, etc. Luminophores can be observed in action in fluorescent lights, TV screens, computer monitor screens, organic light-emitting diodes and bioluminescence.

A chromophore is a region in a molecule where the energy difference between two different molecular orbitals falls within the range of the visible spectrum. Visible light that hits the chromophore can thus be absorbed by exciting an electron from its ground state into an excited state. In biological molecules that serve to capture or detect light energy, the chromophore is the moiety that causes a conformational change of the molecule when hit by light. Chromophores almost always arise in one of two forms: conjugated pi systems and metal complexes. In the former, the energy levels that the electrons jump between are extended pi orbitals created by a series of alternating single and double bonds, often in aromatic systems. Common examples include retinal (used in the eye to detect light), various food colorings, fabric dyes (azo compounds), lycopene, β-carotene, and anthocyanins. The metal complex chromophores arise from the splitting of d-orbitals by binding of a transition metal to ligands. Examples of such chromophores can be seen in chlorophyll (used by plants for photosynthesis), hemoglobin, hemocyanin, and colorful minerals such as malachite and amethyst.

A fluorophore, in analogy to a chromophore, is a component of a molecule which causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. This technology has particular importance in the field of biochemistry and protein studies, e.g., in immunofluorescence and immunohistochemistry. Fluorescein isothiocyanate (FITC), a reactive derivative of fluorescein, has been one of the most common fluorophores chemically attached to other, non-fluorescent molecules to create new fluorescent molecules for a variety of applications. Other historically common fluorophores are derivatives of rhodamine (TRITC), coumarin, and cyanine. Newer generations of fluorophores such as the CF Dyes, the FluoProbes, the DyLight Fluors, the Oyester(dyes), the Atto dyes, the HiLyte Fluors, and the Alexa Fluors that are claimed to be perform better (more photostable, brighter, and/or less pH-sensitive) than other standard dyes of comparable excitation and emission.

These fluorophores can be quantum dots, protein (green fluorescent protein) or small molecules. Common small molecule dye families are: xanthene derivatives (fluorescein, rhodamine, Oregon green, eosin, texas red, etc.), cyanine derivatives (cyanine, indocarbocyanine, oxacarbocyanine, thiacarbocyanine, merocyanine, etc.), naphthalene derivatives (dansyl and prodan derivatives), coumarin derivatives, oxadiazole derivatives (pyridyloxazole, nitrobenzoxadiazole, benzoxadiazole, etc.), pyrene derivatives (cascade blue, etc.), BODIPY (Invitrogen), oxazine derivatives (Nile red, Nile blue, cresyl violet, oxazine 170, etc.), acridine derivatives (proflavin, acridine orange, acridine yellow, etc.), arylmethine derivatives (auramine, crystal violet, malachite green, etc.), CF dye (Biotium), Alexa Fluor (Invitrogen), Atto and Tracy (Sigma), Tetrapyrrole derivatives (porphin, phtalocyanine, bilirubin, etc.), and others (cascade yellow, azure B, acridine orange, DAPI, Hoechst 33258, lucifer yellow, piroxicam, quinine and anthraqinone, squarylium, oligophenylenes, etc.).

Phosphors are transition metal compounds or rare earth compounds of various types. The most common uses of phosphors are in CRT displays and fluorescent lights. CRT phosphors were standardized beginning around World War II and designated by the letter “P” followed by a number. A material can emit light either through incandescence, where all atoms radiate, or by luminescence, where only a small fraction of atoms, called emission centers or luminescence centers, emit light. In inorganic phosphors, these inhomogeneities in the crystal structure are created usually by addition of a trace amount of dopants, impurities called activators. (In rare cases dislocations or other crystal defects can play the role of the impurity.) The wavelength emitted by the emission center is dependent on the atom itself, and on the surrounding crystal structure.

The scintillation process in inorganic materials is due to the electronic band structure found in the crystals. An incoming particle can excite an electron from the valence band to either the conduction band or the exciton band (located just below the conduction band and separated from the valence band by an energy gap). This leaves an associated hole behind, in the valence band. Impurities create electronic levels in the forbidden gap. The excitons are loosely bound electron-hole pairs which wander through the crystal lattice until they are captured as a whole by impurity centers. The latter then rapidly de-excite by emitting scintillation light (fast component). In case of inorganic scintillators, the activator impurities are typically chosen so that the emitted light is in the visible range or near-UV where photomultipliers are effective. The holes associated with electrons in the conduction band are independent from the latter. Those holes and electrons are captured successively by impurity centers exciting certain metastable states not accessible to the excitons. The delayed de-excitation of those metastable impurity states, slowed down by reliance on the low-probability forbidden mechanism, again results in light emission (slow component). In one aspect of the present disclosure, the luminophore Carboxytetramethylrhodamine (TAMRA) is used in the methods and kits.

### C. Microarray

Protein and chemical microarrays have emerged as two important tools in the field of proteomics (Xu and Lam, J Biomed Biotechnol (2003) 5: 257-266). Specific proteins, antibodies, small molecule compounds, peptides, and carbohydrates can now be immobilized on solid surfaces to form microarrays, just like DNA microarrays. These arrays of molecules can then be probed with simple composition of molecules or complex analytes.

Interactions between the analytes and the immobilized array of molecules are evaluated with a number of different detection systems. Typically, commercial use of microarrays employs optical detection with fluorescent, chemiluminescent or enzyme labels, electrochemical detection with enzymes, ferrocene or other electroactive labels, as well as label-free detection based on surface plasmon resonance or microgravimetric techniques (Sassolas et al., Chem Rev (2008) 108: 109-139). To further simplify the assay protocol and reduce the reliance on related equipment, magnetic bead labeling was employed so that assay results could be photographed with a charge-coupled device (CCD) assisted camera or viewed under low magnification microscope (Guo et al., J Anal Sci (2007) 23: 1-4; Li et al., supra; Shlyapnikov et al., Anal Biochem (2010) 399: 125-131), and cross-reactive contacts or unspecific bonds even can be quickly eliminated by applying magnetic field or shear flow (Mulvaney et al., Anal Biochem (2009) 392: 139-144). The detection of microarray-hybridized DNA with magnetic beads thus opens a new way to routine hybridization assays which do not require precise measurements of DNA concentration in solution.

Luminophore markers or luminophores are convenient to underline variations in signal intensity or emission spectra resulting from the binding of target/probe molecular complex. In some aspects, luminophore-labeling can be integrated with magnetic beads, facilitating the process of microarray-based assays. Luminophore-labeled molecules can be coupled to magnetic beads, each of which assembles a large amount of lumiphores at the same time, yielding high intensity of luminescence. High sensitivity detection of molecular interaction thus becomes possible.

The main hindrance to improve both sensitivity and specificity of microarray-based assays is that, as hybridization of labeled nucleic acid targets with surface-immobilized oligonucleotide probes is the central event in the detection of nucleic acids on microarrays (Riccelli et al., Nucleic Acids Res (2001) 29: 996-1004), only one of the two strands of DNA products is available to hybridize with these probes while the other one competes with the probes for the targets, acting as a severe interfering factor. Therefore, single-stranded DNA (ssDNA) should be enriched, and considering simplicity and cost-effectiveness, asymmetric polymerase chain reaction (PCR) was recommended in a previous work after comparing several most popular methods, and a one-step asymmetric PCR without purification process was also developed successfully with its enhanced sensitivity and specificity satisfying the requirements (Gao et al., Anal Lett (2003) 33: 2849-2863; Zhu et al., supra; Li et al., supra).

Typically, for rare clinical samples and their extreme importance of accuracy in detection, the one-step asymmetric PCR-based assay is incapable to deal with, due to its low sensitivity. An alternative way is to employ microspheres, preferably paramagnetic microspheres due to their easy handling and good biocompatibility, which can be further improved with the concern of sensitivity (Gao et al., supra). Through capturing double-stranded DNA fragments with microspheres and removing the unwanted strands by denaturation methods, the yielded ssDNA products were hybridized with microarrays. Theoretically, the purer and more abundance the ssDNA products can be made, the better sensitivity is expected to achieve. As the common symmetric PCR has its properties of much higher amplification efficiency and easier design of multiplexing compared with asymmetric PCR, the combination of symmetric PCR and ssDNAs prepared with this method is expected to meet the above requirement.

In a high-throughput manner, microarray technologies enable the evaluation of up to tens of thousands of molecular interactions simultaneously. Microarrays have made significant impact on biology, medicine, drug discovery. DNA microarray-based assays have been widely used, including the applications for gene expression analysis, genotyping for mutations, single nucleotide polymorphisms (SNPs), and short tandem repeats (STRs). And polypeptide and chemical microarrays have emerged as two important tools in the field of proteomics. Chemical microarray, a form of combinatorial libraries, can also be used for lead identification, as well as optimization of these leads. In this era of bioterrorism, the development of a microarray capable of detecting a multitude of biological or chemical agents in the environment will be of great interest to the law enforcement agencies.

According to some embodiments of the present disclosure, assay methods for analysis of molecular interactions are provided. According to some embodiments of the present disclosure, assay methods for multiplexed analysis of target polynucleotides are provided. The inventive technology improves specificity and sensitivity of microarray-based assays while reducing the cost of performing genetic assays.

As those of ordinary skill in the art will recognize, this disclosure has an enormous number of applications, especially in assays and techniques for pharmaceutical development and diagnostics. The assays may be designed, for example, to detect polynucleotide molecules associated with any of a number of infectious or pathogenic agents including fungi, bacteria, mycoplasma, rickettsia, chlamydia, viruses, and protozoa, or to detect polynucleotide fragments associated with sexually transmitted disease, pulmonary disorders, gastrointestinal disorders, cardiovascular disorders, etc.

A microarray is a multiplex technology widely used in molecular biology and medicine. The target molecules which can be analyzed by microarray include polynucleotides, polypeptides, antibodies, small molecule compounds, peptides, and carbohydrates. Microarrays can be fabricated using a variety of technologies, including printing with fine-pointed pins, photolithography using pre-made masks, photolithography using dynamic micromirror devices, ink-jet printing, microcontact printing, or electrochemistry on microelectrode arrays. In standard microarrays, the probe molecules are attached via surface engineering to a solid surface of supporting materials, which include glass, silicon, plastic, hydrogels, agaroses, nitrocellulose and nylon.

For DNA microarray, it comprises or consists of an arrayed series of microscopic spots of DNA oligonucleotides, known as probes. This can be a short section of a gene or other DNA element that are used to hybridize a complementary polynucleotide sample (called target) under stringent conditions. Targets in solution are usually detected and quantified by detection of fluorophore-, silver-, or chemiluminescence-labeled targets hybridized on microarray. Since an array can contain several to tens of thousands of probes, a microarray experiment can accomplish many genetic tests in parallel.

The systems described herein may comprise two or more probes that detect the same target polynucleotide. For example, in some embodiments where the system is a microarray, the probes may be present in multiple (such as any of 2, 3, 4, 5, 6, 7, or more) copies on the microarray. In some embodiments, the system comprises different probes that detect the same target polynucleotide. For example, these probes may bind to different (overlapping or non-overlapping) regions of the target polynucleotide.

Any probes that are capable of determining the levels of target polynucleotide can be used. In some embodiments, the probe may be an oligonucleotide. It is understood that, for detection of target polynucleotides, certain sequence variations are acceptable. Thus, the sequence of the oligonucleotides (or their complementary sequences) may be slightly different from those of the target polynucleotides described herein. Such sequence variations are understood by those of ordinary skill in the art to be variations in the sequence that do not significantly affect the ability of the oligonucleotide to determine target polynucleotide levels. For example, homologs and variants of these oligonucleotide molecules possess a relatively high degree of sequence identity when aligned using standard methods. Oligonucleotide sequences encompassed by the present disclosure have at least 40%, including for example at least about any of 50%, 60%, 70%, 80%, 90%, 95%, or more sequence identity to the sequence of the target polynucleotides described herein. In some embodiments, the oligonucleotide comprises a portion for detecting the target polynucleotides and another portion. Such other portion may be used, for example, for attaching the oligonucleotides to a substrate. In some embodiments, the other portion comprises a non-specific sequence (such as poly-T or poly-dT) for increasing the distance between the complementary sequence portion and the surface of the substrate.

The oligonucleotides for the systems described herein include, for example, DNA, RNA, PNA, ZNA, LNA, combinations thereof, and/or modified forms thereof. They may also include a modified oligonucleotide backbone. In some embodiments, the oligonucleotide comprises at least about any of 9, 10, 12, 13, 14, 15, 16, 17, 18, 19, 20, or more continuous oligonucleotides complementary or identical to all or part of target polynucleotides described herein. A single oligonucleotide may comprise two or more such complementary sequences. In some embodiments, there may be a reactive group (such as an amine) attached to the 5′ or 3′ end of the oligonucleotide for attaching the oligonucleotide to a substrate.

In some embodiments, the probes are oligonucleotides. Oligonucleotides forming the array may be attached to the substrate by any number of ways including, but not limiting to, (i) in situ synthesis (e.g., high-density oligonucleotide arrays) using photolithographic techniques; (ii) spotting/printing at medium to low density on glass, silicon, nylon or nitrocellulose; (iii) masking; and (iv) dot-blotting on a nylon or nitrocellulose hybridization membrane. Oligonucleotides may also be non-covalently immobilized on the substrate by binding to anchors in a fluid phase such as in microtiter wells, microchannels or capillaries.

Several techniques are well-known in the art for attaching polynucleotides to a solid substrate such as a glass slide. One method is to incorporate modified bases or analogs that contain a moiety that is capable of attachment to a solid substrate, such as an amine group, a derivative of an amine group or another group with a positive charge, into the amplified polynucleotides. The amplified product is then contacted with a solid substrate, such as a glass slide, which may be coated with an aldehyde or another reactive group which can form a covalent link with the reactive group that is on the amplified product and become covalently attached to the glass slide. Microarrays comprising the amplified products can be fabricated using a Biodot (BioDot, Inc. Irvine, Calif.) spotting apparatus and aldehyde-coated glass slides (CEL Associates, Houston, Tex.). Amplification products can be spotted onto the aldehyde-coated slides, and processed according to published procedures (Schena et al., Proc. Natl. Acad. Sci. U.S.A. (1995), 93:10614-10619). Arrays can also be printed by robotics onto glass, nylon (Ramsay, G., Nature Biotechnol. (1998), 16:40-44), polypropylene (Matson, et al., Anal Biochem. (1995), 224(1): 110-6), and silicone slides (Marshall and Hodgson, Nature Biotechnol. (1998), 16:27-31). Other approaches to array assembly include fine micropipetting within electric fields (Marshall, and Hodgson, Nature Biotechnol. (1998), 16:27-31), and spotting the polynucleotides directly onto positively coated plates. Methods such as those using amino propyl silicon surface chemistry are also known in the art, as disclosed at http://cmgm.stanford.edu/pbrown/.

The assays of the present disclosure may be implemented in a multiplex format. Multiplex methods are provided employing 2, 3, 4, 5, 10, 15, 20, 25, 50, 100, 200, 500, 1000 or more different capture probes which can be used simultaneously to assay for amplification products from corresponding different target polynucleotides. In some embodiments, multiplex methods can also be used to assay for polynucleotide target sequences which have not undergone an amplification procedure. Methods amenable to multiplexing, such as those taught herein, allow acquisition of greater amounts of information from smaller specimens. The need for smaller specimens increases the ability of an investigator to obtain samples from a larger number of individuals in a population to validate a new assay or simply to acquire data, as less invasive techniques are needed.

Where different substrates are included in a multiplex assay as part of the capture probe conjugates, the different substrates can be encoded so that they can be distinguished. Any encoding scheme can be used; conveniently, the encoding scheme can employ one or more different fluorophores, which can be fluorescent semiconductor nanocrystals. High density spectral coding schemes can be used.

One or more different populations of spectrally encoded capture probe conjugates can be created, each population comprising one or more different capture probes attached to a substrate comprising a known or determinable spectral code comprising one or more semiconductor nanocrystals or fluorescent nanoparticle. Different populations of the conjugates, and thus different assays, can be blended together, and the assay can be performed in the presence of the blended populations. The individual conjugates are scanned for their spectral properties, which allows the spectral code to be decoded and thus identifies the substrate, and therefore the capture probe(s) to which it is attached. Because of the large number of different semiconductor nanocrystals and fluorescent nanoparticles and combinations thereof which can be distinguished, large numbers of different capture probes and amplification products can be simultaneously interrogated.

### D. Particles

The present disclosure provides particles, microparticles or beads, preferably magnetic beads, to be used for the microarray-based assay. Particles or beads can be prepared from a variety of different polymers, including but not limited to polystyrene, cross-linked polystyrene, polyacrylic acid, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers and epoxies. The materials have a variety of different properties with regard to swelling and porosity, which are well understood in the art. Preferably, the beads are in the size range of approximately 10 nanometers to 1 millimeter, preferably 100 nanometers to 10 micrometers, and can be manipulated using normal solution techniques when suspended in a solution. The terms “particle,” “bead,” “sphere,” “microparticle,” “microbead” and “microsphere” are used interchangeably herein. In some aspects, the microspheres in the present disclosure can have a detectable property. Such a detectable property can be, e.g., magnetic property, fluorescence, absorbance, reflectance, scattering and the like.

The suitable chemical compositions for the magnetic particles may be ferromagnetic materials and include rare earth containing materials such as, e.g., iron-cobalt, iron-platinum, samarium-cobalt, neodynium-iron-boride, and the like. Other magnetic materials, e.g., superparamagnetic materials such as iron oxides (Fe₃O₄) may be used as well. Among the preferred magnetic materials are included iron-cobalt as such material is generally easier to magnetize, has a stronger magnetization (about 1.7 Tesla) and is less susceptible to corrosion.

Because of the use of particles, expensive readout devices for results may not be necessary. Particles on the microarray spots can be viewed directly with naked eyes if the sizes in diameters of these spots are larger than 0.03 millimeters. In another way, assay results with any spot sizes, from 0.01 millimeters to 5 millimeters in diameter, can be photographed with an ordinary camera or viewed under an appropriate magnification microscope. Certainly, if particles are modified, such as fluorescent, chemiluminescent and enzyme labels, corresponding methods can be employed, for instance, electrochemical detection with enzymes, ferrocene or other electroactive labels, as well as label-free detection based on surface plasmon resonance or microgravimetric techniques. If possible, commercial fluorescence microarray scanner may be used to detect fluorescence-labeled particles or the particles with their own autofluorescence.

### E. Target Polynucleotide

The polynucleotide target sequence (or “target polynucleotide”) can be single-stranded, double-stranded, or higher order, and can be linear or circular. Exemplary single-stranded target polynucleotides include mRNA, rRNA, tRNA, hnRNA, microRNA, ssRNA or ssDNA viral genomes and viroids, although these polynucleotides may contain internally complementary sequences and significant secondary structure. Exemplary double-stranded target polynucleotides include genomic DNA, mitochondrial DNA, chloroplast DNA, dsRNA or dsDNA viral genomes, plasmids, phages, shRNA (a small hairpin RNA or short hairpin RNA), and siRNA (small/short interfering RNA). The target polynucleotide can be prepared synthetically or purified from a biological source. The target polynucleotide may be purified to remove or diminish one or more undesired components of the sample or to concentrate the target polynucleotide prior to amplification. Conversely, where the target polynucleotide is too concentrated for a particular assay, the target polynucleotide may first be diluted.

Following sample collection and optional nucleic acid extraction and purification, the nucleic acid portion of the sample comprising the target polynucleotide can be subjected to one or more preparative treatments. These preparative treatments can include in vitro transcription (IVT), labeling, fragmentation, amplification and other reactions. mRNA can first be treated with reverse transcriptase and a primer, which can be the first primer comprising the target noncomplementary region, to create cDNA prior to detection and/or further amplification; this can be done in vitro with extracted or purified mRNA or in situ, e.g., in cells or tissues affixed to a slide. Nucleic acid amplification increases the copy number of sequences of interest and can be used to incorporate a label into an amplification product produced from the target polynucleotide using a labeled primer or labeled nucleotide. A variety of amplification methods are suitable for use, including the polymerase chain reaction method (PCR), transcription mediated amplification (TMA), the ligase chain reaction (LCR), self sustained sequence replication (3 SR), nucleic acid sequence-based amplification (NASBA), rolling circle amplification (RCA), loop-mediated isothermal amplification (LAMP), the use of Q Beta replicase, reverse transcription, nick translation, and the like, particularly where a labeled amplification product can be produced and utilized in the methods taught herein.

Any nucleotides may be detected by the present devices and methods. Examples of such nucleotides include AMP, GMP, CMP, UMP, ADP, GDP, CDP, UDP, ATP, GTP, CTP, UTP, dAMP, dGMP, dCMP, dTMP, dADP, dGDP, dCDP, dTDP, dATP, dGTP, dCTP and dTTP.

In some embodiments, the target polynucleotide does not have a label directly incorporated in the sequence. When the target polynucleotide target sequence is made with a directly incorporated label or so that a label can be directly bound to the target polynucleotide, this label is one which does not interfere with detection of the capture probe conjugate substrate and/or the report moiety label.

Where the target polynucleotide is single-stranded, the first cycle of amplification forms a primer extension product complementary to the target polynucleotide. If the target polynucleotide is single-stranded RNA, a reverse transcriptase is used in the first amplification to reverse transcribe the RNA to DNA, and additional amplification cycles can be performed to copy the primer extension products. The primers for a PCR must, of course, be designed to hybridize to regions in their corresponding template that will produce an amplifiable segment; thus, each primer must hybridize so that its 3′ nucleotide is base-paired with a nucleotide in its corresponding template strand that is located 3′ from the 3′ nucleotide of the primer used to prime the synthesis of the complementary template strand.

The target polynucleotide may be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity which can copy the target polynucleotide can be used, including DNA polymerases, RNA polymerases, reverse transcriptases, enzymes having more than one type of polymerase activity. The polymerase can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase™ T7, Sequenase™ Version 2.0 T7, Tub, Taq, Tth, Pfx, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H minus MMLV (SuperScript™), SuperScript™ II, ThermoScript™, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfx, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions, optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different regions of a particular polynucleotide within the sample. Where the amplification reaction comprises multiple cycles of amplification with a polymerase, as in PCR, it is desirable to dissociate the primer extension product(s) formed in a given cycle from their template(s). The reaction conditions are therefore altered between cycles to favor such dissociation; typically this is done by elevating the temperature of the reaction mixture, but other reaction conditions can be altered to favor dissociation, for example lowering the salt concentration and/or raising the pH of the solution in which the double-stranded polynucleotide is dissolved. Although it is preferable to perform the dissociation in the amplification reaction mixture, the polynucleotides may be first isolated using any effective technique and transferred to a different solution for dissociation, then reintroduced into an amplification reaction mixture for additional amplification cycles.

This assay can be multiplexed, i.e., multiple distinct assays can be run simultaneously, by using different pairs of primers directed at different targets, which can be unrelated targets, or different alleles or subgroups of alleles from, or chromosomal rearrangements at, the same locus. This allows the quantitation of the presence of multiple target polynucleotides in a sample (e.g., specific genes in a cDNA library). All that is required is an ability to uniquely identify the different second polynucleotide extension products in such an assay, through either a unique capture sequence or a unique label.

Amplified target polynucleotides may be subjected to post-amplification treatments. For example, in some cases, it may be desirable to fragment the amplification products prior to hybridization with a polynucleotide array, in order to provide segments which are more readily accessible and which avoid looping and/or hybridization to multiple capture probes. Fragmentation of the polynucleotides can be carried out by any method producing fragments of a size useful in the assay being performed; suitable physical, chemical and enzymatic methods are known in the art.

Amplified target polynucleotides may also be coupled to the particles, either directly or through modifications to the polynucleotides and/or the particles. In some embodiments, the target polynecleotides are modified, such as biotinylation. In some other embodiments, the particles are modified with a functional group, such as streptavidin, neutravidin, avidin, etc. The target polynucleotides may be coupled to the particles through such modifications and functional groups. For double stranded polynucleotides, following the coupling of the target polynucleotides to the particles, single-stranded target polynucleotides can be prepared by denaturation methods by a chemical reaction, enzyme or heating, or a combination thereof, while coupled to the particles. In some embodiments, the chemical reaction uses urea, formamide, methanol, ethanol, an enzyme, or NaOH. In some embodiments, enzymatic methods include exonuclease and Uracil-N-glycosylase. In some other embodiments, the double-stranded target polynucleotide is heat denatured at an appropriate temperature from about 30° C. to about 95° C.

The method of the present disclosure is suitable for use in a homogeneous multiplex analysis of multiple target polynucleotides in a sample. Multiple target polynucleotides can be generated by amplification of a sample by multiple amplification oligonucleotide primers or sets of primers, each primer or set of primers specific for amplifying a particular polynucleotide target sequence. For example, a sample can be analyzed for the presence of multiple viral polynucleotide target sequences by amplification with primers specific for amplification of each of multiple viral target sequences, including, e.g., human immunodeficiency virus (HIV), hepatitis B virus (HBV), hepatitis C virus (HCV), hepatitis A virus (HAV), parvovirus B19, West Nile Virus, hantavirus, severe acute respiratory syndrome-associated coronavirus (SARS), etc.

The portion of the sample comprising or suspected of comprising the target polynucleotide can be any source of biological material which comprises polynucleotides that can be obtained from a living organism directly or indirectly, including cells, tissue or fluid, and the deposits left by that organism, including viruses, mycoplasma, and fossils. The sample can also comprise a target polynucleotide prepared through synthetic means, in whole or in part. Typically, the sample is obtained as or dispersed in a predominantly aqueous medium. Nonlimiting examples of the sample include blood, blood spot (such as dried blood spot), plasma, urine, semen, milk, sputum, mucus, a buccal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, samples of in vitro cell culture constituents (including but not limited to conditioned medium resulting from the growth of cells in cell culture medium, putatively virally infected cells, recombinant cells, and cell components), and a recombinant source, e.g., a library, comprising polynucleotide sequences.

The sample can be a positive control sample which is known to contain the target polynucleotide or a surrogate thereof. A negative control sample can also be used which, although not expected to contain the target polynucleotide, is suspected of containing it, and is tested in order to confirm the lack of contamination by the target polynucleotide of the reagents used in a given assay, as well as to determine whether a given set of assay conditions produces false positives (a positive signal even in the absence of target polynucleotide in the sample).

The sample can be diluted, dissolved, suspended, extracted or otherwise treated to solubilize and/or purify any target polynucleotide present or to render it accessible to reagents which are used in an amplification scheme or to detection reagents. Where the sample contains cells, the cells can be lysed or permeabilized to release the polynucleotides within the cells. Permeabilization buffers can be used to lyse cells which allow further steps to be performed directly after lysis, for example a polymerase chain reaction.

### F. Genetic Information

Any kind of genetic information can be the subject of the presently claimed method of microarray based analysis. For example, the genetic information may be a mutation selected from the group consisting of a substitution, an insertion, a deletion and an indel. In one embodiment, the genetic information is a single nucleotide polymorphism (SNP). In one embodiment, the genetic information is a gene. In one embodiment, the genetic information is a genetic product including a polypeptide, an antibody, a small molecule compound, a peptide and a carbohydrate. In another embodiment, the genetic information is associated with a disease caused by an infectious or pathogenic agent selected from the group consisting of a fungus, a bacterium, a mycoplasma, a rickettsia, a chlamydia, a virus and a protozoa. In yet another embodiment, the genetic information is associated with a sexually transmitted disease, cancer, cerebrovascular disease, heart disease, respiratory disease, coronary heart disease, diabetes, hypertension, Alzheimer's disease, neurodegenerative disease, chronic obstructive pulmonary disease, autoimmune disease, cystic fibrosis, spinal muscular atrophy, beta thalassemia, phenylalanine hydroxylase deficiency, Duchenne muscular dystrophy, or hereditary hearing loss. In still another embodiment, the genetic information is associated with hereditary hearing loss.

In particular embodiments, the genetic information of the present disclosure is one or more mutations of one or more genes associated with hereditary hearing loss, which mutation or mutations can comprise a substitution and/or a deletion associated with hereditary hearing loss.

The allele of the target gene may be caused by single base substitution, insertion, or deletion, or by multiple-base substitution, insertion or deletion, or indel. Furthermore, modifications to nucleotidic units include rearranging, appending, substituting for or otherwise altering functional groups on the purine or pyrimidine base which form hydrogen bonds to a respective complementary pyrimidine or purine. The resultant modified nucleotidic unit optionally may form a base pair with other such modified nucleotidic units but not with A, T, C, G or U. Basic sites may be incorporated which do not prevent the function of the polynucleotide. Some or all of the residues in the polynucleotide can optionally be modified in one or more ways.

Standard A-T and G-C base pairs form under conditions which allow the formation of hydrogen bonds between the N3-H and C4-oxy of thymidine and the Ni and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-O-D-ribofuran-osyl-purine) may be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine) results in a modified nucleotide which will not effectively base pair with guanosine but will form a base pair with isoguanosine (U.S. Pat. No. 5,681,702). Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al. (1993), supra, and Mantsch et al. (1993) Biochem. 14:5593-5601, or by the method described in U.S. Pat. No. 5,780,610. Other normatural base pairs may be synthesized by the method described in Piccirilli et al. (1990) Nature 343:33-37 for the synthesis of 2,6-diaminopyrimidine and its complement (1-methylpyrazolo-[4,3]pyrimidine-5,7-(4H,6H)-dione). Other such modified nucleotidic units which form unique base pairs are known, such as those described in Leach et al. (1992) J. Am. Chem. Soc. 114:3675-3683 and Switzer et al., supra.

A polymorphic region as defined herein is a portion of a genetic locus that is characterized by at least one polymorphic site. A genetic locus is a location on a chromosome which is associated with a gene, a physical feature, or a phenotypic trait. A polymorphic site is a position within a genetic locus at which at least two alternative sequences have been observed in a population. A polymorphic region as defined herein is said to “correspond to” a polymorphic site, that is, the region may be adjacent to the polymorphic site on the 5′ side of the site or on the 3′ side of the site, or alternatively may contain the polymorphic site. A polymorphic region includes both the sense and antisense strands of the polynucleotide comprising the polymorphic site, and may have a length of from about 100 to about 5000 base pairs. For example, a polymorphic region may be all or a portion of a regulatory region such as a promoter, 5′ UTR, 3′ UTR, an intron, an exon, or the like. A polymorphic or allelic variant is a genomic DNA, cDNA, mRNA or polypeptide having a nucleotide or amino acid sequence that comprises a polymorphism. A polymorphism is a sequence variation observed at a polymorphic site, including nucleotide substitutions (single nucleotide polymorphisms or SNPs), insertions, deletions, indels and microsatellites. Polymorphisms may or may not result in detectable differences in gene expression, protein structure, or protein function. Preferably, a polymorphic region of the present disclosure has a length of about 1000 base pairs. More preferably, a polymorphic region of the disclosure has a length of about 500 base pairs.

Most preferably, a polymorphic region of the disclosure has a length of about 200 base pairs.

A haplotype as defined herein is a representation of the combination of polymorphic variants in a defined region within a genetic locus on one of the chromosomes in a chromosome pair. A genotype as used herein is a representation of the polymorphic variants present at a polymorphic site.

Those of ordinary skill will recognize that oligonucleotides complementary to the polymorphic regions described herein must be capable of hybridizing to the polymorphic regions under conditions of stringency such as those employed in primer extension-based sequence determination methods, restriction site analysis, nucleic acid amplification methods, ligase-based sequencing methods, mismatch-based sequence determination methods, microarray-based sequence determination methods, and the like.

Congenital hearing loss affects one in 1,000 live births and approximately 50% of these cases are hereditary. Mutations in GJB2, GJB6, SLC26A4 and 12S rRNA are the prevalent causes of inherited hearing loss. In many European countries, the prevalence of heterozygotes for GJB2-35delG has been estimated to 2-4% of the population with normal hearing. It has been reported that c.167delT, the deletion T of 167 in the coding region causing a frameshift mutation, is the most prevalent mutation in Jewish people. The mutation W44C is associated with dominantly inherited NSAHI. Further analysis demonstrated that some GJB2 heterozygotes also carried a truncating deletion of the GJB6 gene, encoding connexin 30, in trans. The common 309 kb deletion, involving the coding region GJB6 gene upstream of GJB2 gene has been identified and found to account for up to 10% of DFNB1 alleles in Caucasians. Several studies showed that MT-RNR1 gene mutations, such as the m.1555A>G can also cause hearing loss. There are other MT-RNR1 gene mutations associated with HL, such as m.7444G>A. G7444A is a common variant within the West European prevalent haplogroup. Apart from mutations in GJB2 which are the most common cause of NSHL, variants in SLC26A4 are the second most cause of NSHL according to some reports. To date, more than 170 variations in SLC26A4 have been reported. Among them, the most common mutations in Caucasian population are L236P. The most common mutations of SLC26A4 gene are p.T416P in Northern Europe. In some aspects, the present disclosure meets the need of nine mutation detection from various deafness patients or even healthy persons of Caucasian populations, which also serves as an example to support the applicability of the presently disclosed technology.

### G. Oligonucleotide Primers for Amplification of Target Polynucleotides

In certain aspect, the disclosure is also embodied in oligonucleotide primer pairs suitable for use in the polymerase chain reaction (PCR) or in other nucleic acid amplification methods. Those of ordinary skill will be able to design suitable oligonucleotide primer pairs using knowledge readily available in the art, in combination with the teachings herein. Specific oligonucleotide primer pairs of this embodiment include the oligonucleotide primer pairs set forth in Table 2, which are suitable for amplifying the polymorphic regions corresponding to polymorphic sites in GJB2, GJB6, SLC26A4 and 12S rRNA. Those of ordinary skill will recognize that other oligonucleotide primer pairs suitable for amplifying the polymorphic regions in GJB2, GJB6, SLC26A4 and 12S rRNA can be designed without undue experimentation.

In some variations a SNP/mutation corresponds to at least two allele-specific primers. One allele-specific primer comprises a sequence identical or complementary to a region of the wild-type allele of a target fragment containing the SNP/mutation locus. Each of the other allele-specific primers comprises a sequence identical or complementary to a region of the mutant allele of a target fragment containing the SNP and/or mutation locus. The allele-specific primers may terminate at their 3′ ends at the SNP/mutation locus. To increase the capability of differentiation between the wild-type and mutant alleles of target genes, an artificial mismatch in the allele-specific primers may be introduced. The artificial mismatch can be a natural base or a nucleotide analog. Each of the PCR primer pairs of the disclosure may be used in any PCR method. For example, a PCR primer pair of the disclosure may be used in the methods disclosed in U.S. Pat. Nos. 4,683,195; 4,683,202, 4,965,188; 5,656,493; 5,998,143; 6,140,054; WO 01/27327; WO 01/27329; and the like. The PCR primer pairs of the disclosure may also be used in any of the commercially available machines that perform PCR, such as any of the GeneAmp® Systems available from Applied Biosystems.

The present primers can comprise any suitable types of nucleic acids, e.g., DNA, RNA, PNA or a derivative thereof. Preferably, the primers comprise a nucleotide sequence, or a complementary strand thereof, that is set forth in Table 2. Also preferably, the primers are labeled, e.g., a chemical, an enzymatic, an immunogenic, a radioactive, a fluorescent, a luminescent, and/or a FRET label.

The oligonucleotide primers can be produced by any suitable method. For example, the primers can be chemically synthesized (See generally, Ausubel (Ed.) Current Protocols in Molecular Biology, 2.11. Synthesis and purification of oligonucleotides, John Wiley & Sons, Inc. (2000)), isolated from a natural source, produced by recombinant methods or a combination thereof. Synthetic oligonucleotides can also be prepared by using the triester method of Matteucci et al., J. Am. Chem. Soc., 3:3185-3191 (1981). Alternatively, automated synthesis may be preferred, for example, on an Applied Biosynthesis DNA synthesizer using cyanoethyl phosphoramidite chemistry. Preferably, the primers are chemically synthesized.

Suitable bases for preparing the oligonucleotide primers of the present disclosure may be selected from naturally occurring nucleotide bases such as adenine, cytosine, guanine, uracil, and thymine. It may also be selected from nonnaturally occurring or “synthetic” nucleotide bases such as 8-oxo-guanine, 6-mercaptoguanine, 4-acetylcytidine, 5-(carboxyhydroxyethyl) uridine, 2′-O-methylcytidine, 5-carboxymethylamino-methyl-2-thioridine, 5-carboxymethylaminomethyl uridine, dihydrouridine, 2′-O-methylpseudouridine, beta-D-galactosylqueosine, 2′-Omethylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl) threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl) carbamoyl) threonine, 2′-O-methyl-5-methyluridine, 2′-O-methyluridine, wybutosine, and 3-(3-amino-3-carboxypropyl) uridine.

Likewise, chemical analogs of oligonucleotides (e.g., oligonucleotides in which the phosphodiester bonds have been modified, e.g., to the methylphosphonate, the phosphotriester, the phosphorothioate, the phosphorodithioate, or the phosphoramidate) may also be employed. Protection from degradation can be achieved by use of a “3′-end cap” strategy by which nuclease-resistant linkages are substituted for phosphodiester linkages at the 3′ end of the oligonucleotide (Shaw et al., Nucleic Acids Res., 19:747 (1991)). Phosphoramidates, phosphorothioates, and methylphosphonate linkages all function adequately in this manner. More extensive modification of the phosphodiester backbone has been shown to impart stability and may allow for enhanced affinity and increased cellular permeation of oligonucleotides (Milligan et al., J. Med. Chem., 36:1923 (1993)). Many different chemical strategies have been employed to replace the entire phosphodiester backbone with novel linkages. Backbone analogues include phosphorothioate, phosphorodithioate, methylphosphonate, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene (methylimino) (MMI) or methyleneoxy (methylimino) (MOMI) linkages. Phosphorothioate and methylphosphonate-modified oligonucleotides are particularly preferred due to their availability through automated oligonucleotide synthesis. The oligonucleotide may be a “peptide nucleic acid” such as described by (Milligan et al., J. Med. Chem., 36:1923 (1993)). The only requirement is that the oligonucleotide primer should possess a sequence at least a portion of which is capable of binding to a portion of a target sequence.

The target polynucleotide may be double stranded or single stranded. In some embodiments, at least a portion of the single-stranded target polynucleotide is completely or substantially complementary to at least a portion of the oligonucleotide probe immobilized on the microarray. In other embodiments, the single-stranded target polynucleotide is completely complementary to the oligonucleotide probe immobilized on the microarray.

Employing PCR, RT-PCR (for RNA molecules) or other methods, polynucleotide molecules/agents of interest can be converted to nucleic acid fragments and labeled with biotin, digoxin or the similar, which then binds with moieties on the surface of particles/beads. By coupling to the particles or beads, these nucleic acid fragments in solution are enriched. For double-stranded nucleic acid fragments, they are denatured to single-stranded ones. Beads are then coupled to specific microarray spots through target-probe hybridization, which directly or through further modifications, facilitate the detection of results with non-expensive devices or common commercial microarray scanners. Specific genes, SNPs or gene mutations, such as deletions, insertions, and indels, are thus identified. For SNPs/mutations, they are valuable for biomedical research and for developing pharmaceutical compounds or medical diagnostics. SNPs are also evolutionarily stable—not changing much from generation to generation—making them convenient to follow in population studies.

Any method may be used to assay the polynucleotide, that is, to determine the polymorphic sites, in this step of the disclosure. For example, any of the primer extension-based methods, ligase-based sequence determination methods, mismatch-based sequence determination methods, or microarray-based sequence determination methods described above may be used, in accordance with the present disclosure. Alternatively, such methods as restriction fragment length polymorphism (RFLP) detection, single strand conformation polymorphism detection (SSCP), denaturing gradient gel electrophoresis (DGGE), denaturing high-performance liquid chromatography (DHPLC), PCR-based assays such as the Taqman® PCR System (Applied Biosystems) may be used.

Allele-specific PCR (ASPCR) is known as amplification refractory mutation system (ARMS) or PCR-sequence specific primer (PCR-SSP), etc. With high accuracy, ASPCR is suitable for analyzing known SNPs/mutations in genetic sequences, which uses DNA polymerase without the 3′-5′ exonuclease activity so that if the 3′ end of a specific primer does not match the template, the primer can not be elongated and the PCR reaction is blocked. Utilizing multiplex PCR, multiple loci can be amplified simultaneously, and then distinguished by DNA microarray. The PCR amplification may be conducted in one tube, or in different tubes.

By employing the universal array technology, Tag sequences are conjugated with primers, and their final products can readily hybridize with the Tag probes. Microarrays here just serve as a decode tool. The Tag sequences are artificially designed and subject to critical filtering, they have the corresponding complementary sequences, cTag sequences. Each combination of Tag and cTag corresponds to an allele of a SNP/mutation in the target gene. The Tm difference between different Tag sequences equals or is less than 5° C., e.g., about 5° C., 4.9° C., 4.8° C., 4.7° C., 4.6° C., 4.5° C., 4.4° C., 4.3° C., 4.2° C., 4.1° C., 4.0° C., 3.5° C., 3.0° C., 2.5° C., 2.0° C., 1.5° C., 1.0° C., or less, and the Tag sequences have no cross-hybridization among themselves or with the group of primers, have low homology to the species of the sample genomic DNA, and no hair-pin structures. Determination of genes or genotypes is based on the hybridization signal and the position of the Tag probes on microarray hybridized with the PCR products.

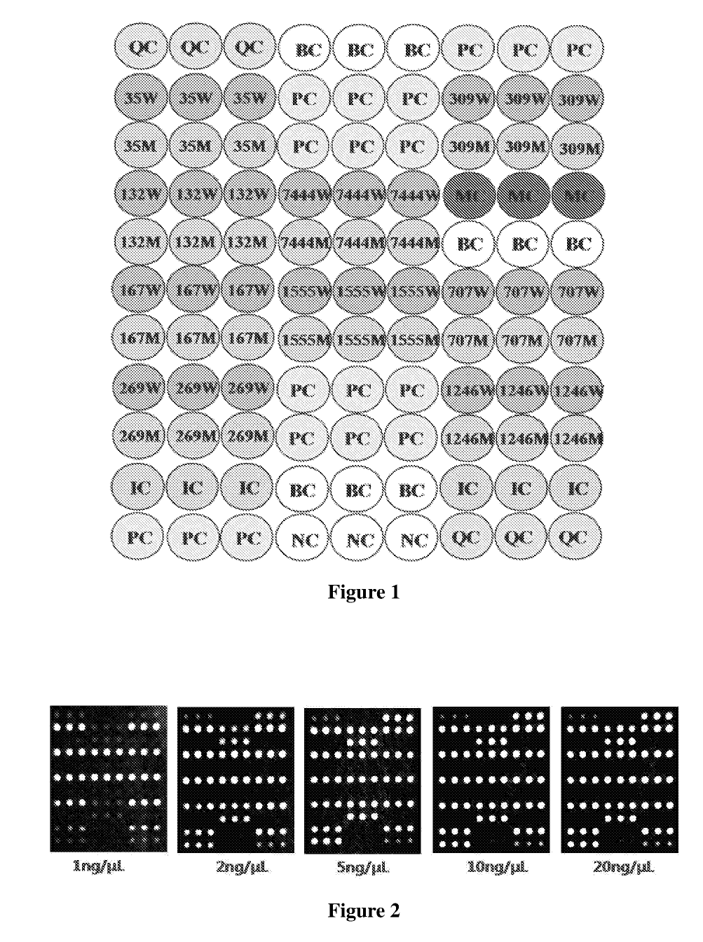

FIG. 1 shows the layout of universal Tag array as an example for de-multiplexing nine mutations of Caucasian populations related to hereditary hearing loss.

Each Tag probe on the universal array comprises a nucleotide sequence of any one of the Tag sequences shown in Table 1. In some variations, each Tag probe is 5′-amino-modified, and comprises a 15-nucleotide poly-dT spacer linked to the 5′ end of the Tag sequences. QC and BC represent positive and negative controls of spotting efficiency, respectively. PC and NC represent positive and negative controls of hybridization, respectively. IC represents positive control of PCR reaction. MC represents positive control of the microsphere surface-modified moieties binding with their targets. These considerations make sure that each step within assay procedure is accurately carried out as well as the final results. Of course, one can use many more or less Tag sequences with or without replicate spots for specific applications. These Tag sequences may be designed by methods of bioinformatics. Tag probes can also be derived from a biological species different from the species of the target gene. For example, if the species of the target is from human, the Tag sequences can be derived from sequences of bacteria. The Tag sequence is single stranded oligonucleotide or peptide oligonucleotide.

The universal array in this disclosure is different from the common microarray. For common microarray, the probes on the array may be gene-specific or allele-specific oligonucleotides. Different target gene panel or SNP/mutation panel needs different format of microarray. However, the universal array in this disclosure consists of Tag probes which are specifically designed, so they are not associated with allele-specific oligonucleotides or primers. The Tag sequences can be used as codes for different SNP/mutation of different genes or different species. One format of universal array can be used for detection of any gene or genotype. So such array is universal and the process of detection is a kind of de-coding step.

### H. Kits

A kit useful for detecting a molecular interaction comprising a particle, a microarray and a probe molecule immobilized on the microarray is hereby provided in this disclosure. In certain aspect, the disclosure is also embodied in a kit comprising a universal Tag array. Preferably, the kit of the disclosure comprises set of primers for ASPCR amplification of a genetic information comprising two allele-specific primers and a common primer as set forth in Table 2. The kit of the disclosure may also comprise a polymerizing agent, for example, a thermostable nucleic acid polymerase such as those disclosed in U.S. Pat. Nos. 4,889,818; 6,077,664, and the like. The kit of the disclosure may also comprise chain elongating nucleotides, such as dATP, dTTP, dGTP, dCTP, and dITP, including analogs of dATP, dTTP, dGTP, dCTP and dITP, so long as such analogs are substrates for a thermostable nucleic acid polymerase and can be incorporated into a growing nucleic acid chain. In a preferred embodiment, the kit of the disclosure comprises at least one oligonucleotide primer pair, a polymerizing agent, and chain elongating nucleotides. The kit of the disclosure may optionally include buffers, vials, microtiter plates, and instructions for use.

### I. Exemplary Embodiments

The following examples are offered to illustrate but not to limit the disclosure.

Samples

Patient blood samples with known mutations associated with hereditary deafness, and samples with unknown mutations including buccal swabs and dried blood spots, were provided by Miami University.

Primers

Multiplex PCR primers used for analyzing a total of 9 mutations are listed in Table 2. In column Mutation Type ‘del’ represents a deletion mutation, e.g., c.35delG means a deletion of G at position 35 in the coding region of GJB2; ‘>’ represents a substitution mutation, e.g. c.132G>C means a substitution of G by C at position 132 in the coding region of GJB2 (Cx26). Primer Name with ‘WT’ or ‘MU’ suffix represents an allele-specific primer capable of specifically amplifying the wild-type or mutant allele at the mutation locus, respectively. Primer Name with a ‘RB’ suffix represent a common primer, biotinylated at the 5′-termini, capable of amplifying both the wild-type allele and the mutant allele of the target genetic fragments including the mutation locus. The common primer is also fluorescence-labeled with Cy3-dTTP while synthesized, which is asterisked in Table 2. For each mutation locus the two allele-specific primers respectively pair with the common primer. ‘In order to improve assay specificity, artificial mismatches (underlined) are introduced into some of the allele-specific primers.

Probes

The universal array is a matrix made up of 18 Tag probes capable of hybridizing to the multiplex PCR products, besides positive quality control for sample spotting (QC), negative quality control for sample spotting (BC), positive quality control for hybridization (PC), positive quality control for PCR (IC), negative quality control for hybridization (NC), and positive control of the streptavidin-coated particles binding with biotin-labeled DNA fragments (MC). QC is an oligonucleotide probe labeled with fluorescence HEX at one end and modified by an amino group (NH₂) at the other end to monitor the efficacy of sample spotting and fixing on the array. BC is a spotting buffer for quality control of cross contamination during sample spotting. NC is an oligonucleotide probe modified by an amino group which is theoretically incapable of hybridizing to any fragment in solution for quality control of nonspecific hybridization.

PC is an oligonucleotide probe modified by an amino group which is quality control of hybridization. IC is an oligonucleotide probe modified by an amino group which is capable of hybridizing to the house keeping gene products for quality control of PCR. MC is an oligonucleotide probe modified by an amino group and biotinylated for quality control of the streptavidin-coated particles binding with biotinylated DNA fragments.

The Tag probes on the universal array are designed according to the format: NH₂-TTTTTTTTTTTTTTT-TagX, where X is a natural number between 1 and 18. The Tag probes have a 5′-amino group modification, followed by poly-dT15, followed by Tag1 to Tag18 with the sequences 1 to 18 listed in Table 1, respectively. The nucleotide sequences of Tag1 to Tag18 in the Tag probes are identical to the corresponding sequences of Tag1 to Tag18 of the primers, respectively.

Multiplex Allele-Specific PCR

Multiplex PCR was carried out using the genomic DNA extracted from whole blood samples and dried blood spots from patients or high risk family for deafness as templates. Reaction volumes were 25 μL, and contained 0.2 mM dNTPs, 0.1 mM dUTP, 1× Qiagen PCR buffer, with addition of MgCl₂ to 2 mM, 1 unit of HotStartTaq DNA polymerase lacking of a 3′ to 5′ exonuclease activity (Qiagen, Hilden, Germany) and 10 ng of genomic DNA, and 0.03-0.91 μM primers for each selected mutation. For determining the assay detection limit, different quantities of genomic DNA were used, ranging from 2 ng to 20 ng. Amplification was performed in a PTC-225 Thermal Cycler (MJ Research, Watertown, Mass.). Amplification program was as follows: first 95° C. for 15 min; then 96° C. for 1 min, ramp at 0.4° C./second down to 55° C., hold at 55° C. for 30 seconds, ramp at 0.2° C./second up to 70° C., hold at 70° C. for 45 seconds, repeat for 32 cycles; finally hold at 60° C. for 10 minutes; and 4° C. soak.

Single-Stranded DNA Isolation

Streptavidin-coated MyOne Dynal beads (Invitrogen Dynal AS, Oslo, Norway) were used, which could capture the biotin-labeled PCR products. These beads were first pretreated according to the protocol from the supplier, and 8 μL of beads were added to 40 μL Binding buffer, incubating for 15 seconds. The removing the solution, and adding 40 μL fresh Binding buffer. Then two washes with binding and washing buffer (5 mM Tris-HCl pH 7.5, 0.5 mM EDTA, 1 M NaCl) were followed. Alkaline denaturation was performed twice with 60 μL freshly prepared 0.1 N NaOH for 10 minutes each time. After that, 33 μL hybridization buffer (9×SSC, 7.5×Denhardt's, 37.5% (v/v) Formamide, 0.15% SDS) was added.

Universal Array Hybridization

The hybridization mixture was added to the surface of universal Tag array. The slides were incubated at 55° C. for 20 minutes and washed 2 minutes each at 42° C. in the washing solution (0.03×SSC), then open laser to capture the picture. The whole progress is done by Easyarray station (CapitalBio, Beijing, China), and the data of obtained images were extracted with SpotData software (CapitalBio) for further analysis. Laser power and photomultiplier tube (PMT) index were 70% and 700, respectively.

### Multiplexed Analysis of Nine Mutations Related to Hereditary Hearing Loss of Caucasian Populations

Microarray-based assay integrated with paramagnetic microspheres was used for multiplexed analysis of nine mutations related to hereditary hearing loss of Caucasian populations. Commercial fluorescent scanner was employed to detect the results, which were accomplished by enriching multiple PCR products with microspheres, harvesting ssDNA fragments, coupling microspheres to universal Tag array through hybridization, and decoding them with the universal Tag array.

FIG. 1 shows, as an example, the layout of universal Tag array corresponding to eight SNPs/mutations related to hereditary hearing loss, where mutations in GJB2 (Cx26) gene, GJB6 (Cx30) gene, SLC26A4 (PDS) gene, and 12S rRNA (MTRNR1) gene were selected. Name with ‘W’ or ‘M’ suffix represents the probe corresponding to the wild-type or mutant allele at the mutation locus, respectively. On the left of the array are probes for wild-type alleles, on the right are probes for mutant alleles, and each probe is printed horizontally as three replica spots. For detecting c.35delG, c.167delT, c.132G>C, and c. 269T>C in the GJB2 (Cx26) gene, c.707T>C and c.1246A>C in the SLC26A4 (PDS) gene, and m.1555A>G and m.7444G>A in 12S rRNA gene (MTRNR1, belonging to mitochondria gene), the primers for each mutation may include two allele-specific primers and one common primer labeled with biotin as well as Cy3, as shown in Table 2. Each allele-specific primer comprises a unique Tag sequence linked to the 5′ end of a nucleotide sequence which is identical or complementary to a target gene sequence containing the /mutation locus. And each allele-specific primer along with common primer generates a DNA fragment containing the mutation locus through PCR amplifications. The probes comprising sequences identical to their corresponding Tag sequences in allele-specific primers are immobilized on a solid surface to form the universal array. Streptavidin-coated particles can be used to capture biotin-labeled DNA products, and after harvesting of ssDNAs the target-probe hybridization is carried out. The results can be interrogated by the fluorescence intensity of coupled particles and the position of corresponding Tag probe on the array.

To determine the assay detection limit, different quantities of genomic DNA from clinical samples, with all wild-type alleles at nine selected SNP/mutation loci, were used, ranging from 2 ng to 20 ng. As shown in FIG. 2, nine selected SNPs/mutations related to hereditary hearing loss of Caucasian populations were simultaneously analyzed, and according to the layout of the universal Tag array schemed in FIG. 1, all the wild-type-specific probes on the left of the array showed positive signal while almost no hybridization signal was detected from mutant-specific probes on the right, indicating that the current detection limit of this application of the invention was 2 ng of genomic DNA.

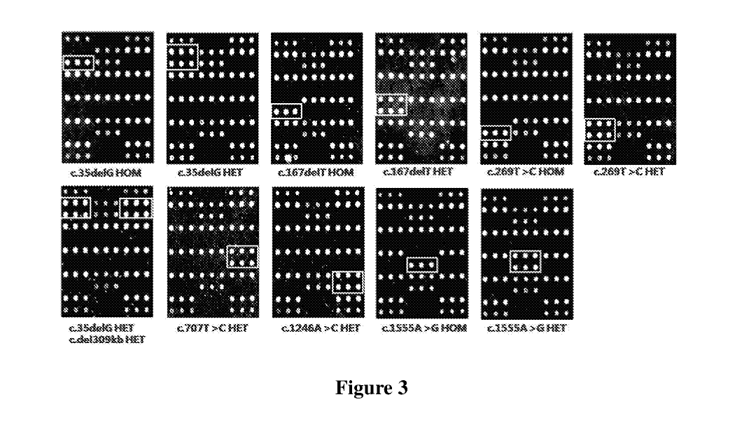

Besides the wild-type, mutant alleles related to nine selected mutations from homozygous and heterozygous clinical samples were examined, as shown in FIG. 3. Within the range from 2 ng to 20 ng, any amount of genomic DNA was suitable for this assay. ‘MU’ and ‘HET’ suffix represent the homozygote and heterozygote, respectively. For heterozygous samples, they contain both wild-type and mutant alleles at a SNP/mutation site. For the SNP/mutation sites in the mitochondria genes such as m.1555A>G, ‘HOM’ and ‘HET’ suffix represent homoplasmic and heteroplasmic mutation state, respectively. With the confirmation of other genotyping methods such as DNA sequencing, the results were of 100% accuracy, demonstrating that such platform had high specificity and was capable of genotyping clinical samples. With the extremely high sensitivity of this genotyping platform, one also can apply it to detect rare samples. In practice, buccal swabs and dried blood spots from families affected by deafness were collected, and their assay results were 100% correct, as confirmed by DNA sequencing. The successful genotyping paves the way for this genotyping platform widely applied in genetic and diagnostic analysis associated with a large number of diseases as well as their corresponding mutations.

TABLE 1
The probes of the universal Tag array

| Name | Sequence (5′→3′) |
|---|---|
| Tag-1 | NH₂-T₁₅-GAGGAGATCGTAGCTGGTGCAT |
| Tag-2 | NH₂-T₁₅-TCGCTGCCAACCGAGAATTGCA |
| Tag-3 | NH₂-T₁₅-CAAGGCACGTCCCAGACGCATCAA |
| Tag-4 | NH₂-T₁₅-GACCGAGGATAGCGTTACCG |
| Tag-5 | NH₂-T₁₅-CTAACGGAGAGACGCAACGG |
| Tag-6 | NH₂-T₁₅-GGTATCGCGACCGCATCCCAATCT |
| Tag-7 | NH₂-T₁₅-GGACGGTAATCTCGCTTCGC |
| Tag-8 | NH₂-T₁₅-GTTAGGGTCGCGCCAAACTCTCC |
| Tag-9 | NH₂-T₁₅-TGCACGAGTTGGGTGAGTTTGG |
| Tag-10 | NH₂-T₁₅-TCGAGTACAGGACCCACATCAG |
| Tag-11 | NH₂-T₁₅-CTGTTAAACGTCAGAGCGCAGC |
| Tag-12 | NH₂-T₁₅-AGTCGAAGTGTGCGTCAGACTC |
| Tag-13 | NH₂-T₁₅-CGAGACGACTTAGGACGAGG |
| Tag-14 | NH₂-T₁₅-ATGACGACCTGAGTGCACACAC |
| Tag-15 | NH₂-T₁₅-GAGCAAGCGCAAACGCAGTACT |
| Tag-16 | NH₂-T₁₅-GCATAGACGTGGCTCAACTGTC |
| Tag-17 | NH₂-T₁₅-ACCTTTCGCTTCACCGGCCGATC |
| Tag-18 | NH₂-T₁₅-GCTCGAAGAGGCGCTACAGATCC |
| MC | NH2-polyT15-GCAACCACCACCGGAGG-Biotin |
| PC | NH2-polyT15-TCCTCAACGGAAGCAAGTGAT-Biotin |
| IC | NH2-polyT15-GCGCTTCAGGGCCCTGTTC |
| NC | NH2-polyT15-GCTTTATCCCTAACGTCATCGGG |
| QC | HEX-polyT15-CAGAGTGCTTGGTGCCATAAC-NH₂ |

TABLE 2
SNPs/Mutations and
their specially designed primers

| Mutation | | |
|---|---|---|
| Type | Primer Name | Primer Sequence (5′→3′) |
| c.35delG | t35delG-WT | Tag1-TGTTTGTTCACACCCCCg |
| | | AG |
| | t35delG-MU | Tag2-TGTTTGTTCACACCCgCA |
| | | G |
| | GJB2-RB-1 | Biotin-GCATGCTTGC*TTACC |
| | | CAGAC |
| c.132G > C | t132G > C-WT | Tag3-AGTCGGCCTGCTCATCTt |
| | | CC |
| | t132G > C-MU | Tag4-ACCCTGCAGCCAGCTACG |
| | GJB2-RB-1 | Biotin-GCATGCTTGC*TTACC |
| | | CAGAC |
| c.167delT | t167delT-WT | Tag5-GACTTTGTCTGCAACACa |
| | | CTGC |
| | t167delT-MU | Tag6-GACTTTGTCTGCAACAaC |
| | | CGC |
| | GJB2-RB-2 | Biotin-TCCCTCTCA*TGCTGT |
| | | CTATTTCTTA |
| c.269T > C | t269T > C-WT | Tag7-TCCACGCCAGCGCaCCT |
| | t269T > C-MU | Tag8-GTCCACGCCAGCGCTCtC |
| | GJB2-RB-2 | Biotin-TCCCTCTCA*TGCTGT |
| | | CTATTTCTTA |
| c.del309kb | tdel309kb-WT | Tag9-CCTACTACAGGCACGAAA |
| | | CCAC |
| | tdel309kb-MU | Tag10-CACCATGCGTAGCCTTA |
| | | ACCATTTT |
| | 309-RB | Biotin-GCACAACTC*TGCCAC |
| | | GTTAAGC |
| c.707T > C | t707T > C-WT | Tag11-cTTGAAACATTGAGGAC |
| | | AATCTaTA |
| | t707T > C-MU | Tag12-GTTGAAACATTGAGGAC |
| | | AATCTgTG |
| | 707-RB | Biotin-CAGAGAGTAGGT*TTC |
| | | TATCTCAGGC |
| c.1246A > C | t1246A > C-WT | Tag13-TTCCTACCTGTGTCTTT |
| | | CCTCaAGT |
| | t1246A > C-MU | Tag14-TCCTACCTGTGTCTTTC |
| | | CTCCtGG |
| | 1246-RB | Biotin-CGTTGTCA*TCCAGTC |
| | | TCTTCCTTAG |
| m.7444G > A | t7444G > A-WT | Tag15-AGAACCCGTATACATAA |
| | | AATCgAG |
| | t7444G > A-MU | Tag16-AGAACCCGTATACATAA |
| | | AATCgAA |
| | 7444-RB | Biotin-GTTAGGAAAAGGGCA* |
| | | TACAGG |
| m.1555A > G | t1555A > G-WT | Tag17-ACTTACCATGTTACGAC |
| | | TaGT |
| | t1555A > G-MU | Tag18-CACTTACCATGTTACGA |
| | | CTcGC |
| | 1555-RB | Biotin-CCCTGA*TGAAGGCTA |
| | | CAAAG |

TABLE 3
Sequences and SEQ ID NOs:

| SEQ ID NO | Sequence |
|---|---|
| SEQ ID NO: 1 | GAGGAGATCGTAGCTGGTGCAT |
| SEQ ID NO: 2 | TCGCTGCCAACCGAGAATTGCA |
| SEQ ID NO: 3 | CAAGGCACGTCCCAGACGCATCAA |
| SEQ ID NO: 4 | GACCGAGGATAGCGTTACCG |
| SEQ ID NO: 5 | CTAACGGAGAGACGCAACGG |
| SEQ ID NO: 6 | GGTATCGCGACCGCATCCCAATCT |
| SEQ ID NO: 7 | GGACGGTAATCTCGCTTCGC |
| SEQ ID NO: 8 | GTTAGGGTCGCGCCAAACTCTCC |
| SEQ ID NO: 9 | TGCACGAGTTGGGTGAGTTTGG |
| SEQ ID NO: 10 | TCGAGTACAGGACCCACATCAG |
| SEQ ID NO: 11 | CTGTTAAACGTCAGAGCGCAGC |
| SEQ ID NO: 12 | AGTCGAAGTGTGCGTCAGACTC |
| SEQ ID NO: 13 | CGAGACGACTTAGGACGAGG |
| SEQ ID NO: 14 | ATGACGACCTGAGTGCACACAC |
| SEQ ID NO: 15 | GAGCAAGCGCAAACGCAGTACT |
| SEQ ID NO: 16 | GCATAGACGTGGCTCAACTGTC |
| SEQ ID NO: 17 | ACCTTTCGCTTCACCGGCCGATC |
| SEQ ID NO: 18 | GCTCGAAGAGGCGCTACAGATCC |
| SEQ ID NO: 19 | GCAACCACCACCGGAGG |
| SEQ ID NO: 20 | TCCTCAACGGAAGCAAGTGAT |
| SEQ ID NO: 21 | GCGCTTCAGGGCCCTGTTC |
| SEQ ID NO: 22 | GCTTTATCCCTAACGTCATCGGG |
| SEQ ID NO: 23 | CAGAGTGCTTGGTGCCATAAC |
| SEQ ID NO: 24 | TGTTTGTTCACACCCCCgAG |
| SEQ ID NO: 25 | TGTTTGTTCACACCCgCAG |
| SEQ ID NO: 26 | GCATGCTTGCTTACCCAGAC |
| SEQ ID NO: 27 | AGTCGGCCTGCTCATCTtCC |
| SEQ ID NO: 28 | ACCCTGCAGCCAGCTACG |
| SEQ ID NO: 29 | GCATGCTTGCTTACCCAGAC |
| SEQ ID NO: 30 | GACTTTGTCTGCAACACaCTGC |
| SEQ ID NO: 31 | GACTTTGTCTGCAACAaCCGC |
| SEQ ID NO: 32 | TCCCTCTCATGCTGTCTATTTCTTA |
| SEQ ID NO: 33 | TCCACGCCAGCGCaCCT |
| SEQ ID NO: 34 | GTCCACGCCAGCGCTCtC |
| SEQ ID NO: 35 | TCCCTCTCATGCTGTCTATTTCTTA |
| SEQ ID NO: 36 | CCTACTACAGGCACGAAACCAC |
| SEQ ID NO: 37 | CACCATGCGTAGCCTTAACCATTTT |
| SEQ ID NO: 38 | GCACAACTCTGCCACGTTAAGC |
| SEQ ID NO: 39 | cTTGAAACATTGAGGACAATCTaTA |
| SEQ ID NO: 40 | GTTGAAACATTGAGGACAATCTgTG |
| SEQ ID NO: 41 | CAGAGAGTAGGTTTCTATCTCAGGC |
| SEQ ID NO: 42 | TTCCTACCTGTGTCTTTCCTCaAGT |
| SEQ ID NO: 43 | TCCTACCTGTGTCTTTCCTCCtGG |
| SEQ ID NO: 44 | CGTTGTCATCCAGTCTCTTCCTTAG |
| SEQ ID NO: 45 | AGAACCCGTATACATAAAATCgAG |
| SEQ ID NO: 46 | AGAACCCGTATACATAAAATCgAA |
| SEQ ID NO: 47 | GTTAGGAAAAGGGCATACAGG |
| SEQ ID NO: 48 | ACTTACCATGTTACGACTaGT |
| SEQ ID NO: 49 | CACTTACCATGTTACGACTcGC |
| SEQ ID NO: 50 | CCCTGATGAAGGCTACAAAG |

What is claimed is:

1. A method for detecting a target molecule using a microarray, which method comprises:
(a) labeling the target molecule with a luminophore;
(b) coupling the target molecule to a particle;
(c) binding the target molecule to a probe molecule immobilized on the microarray; and
(d) detecting the interaction between the target molecule and the probe molecule,
wherein the target molecule comprises a polynucleotide comprising a genetic information which comprises:
(1) a genetic information within the target gene of GJB6 (Cx30); and/or
(2) c.132G>C and/or c.269T>C within the target gene of GJB2; and/or
(3) c.1246A>C within the target gene of SLC26A4; and/or
(4) m.7444G>A within the target gene of 12S rRNA.

2. The method of claim 1, wherein the genetic information within the target gene of GJB6 (Cx30) is c.del309kb.

3. The method of claim 1 or 2, wherein the polynucleotide further comprises one or more of the following genetic information: c.35delG within the target gene of GJB2; c.167delT within the target gene of GJB2; c.707T>C within the target gene of SLC26A4; and m.1555A>G within the target gene of 12S rRNA.

4. The method of any one of claims 1-3, wherein the particle is a microparticle.

5. The method of claim 4, wherein the microparticle is a paramagnetic microsphere.

6. The method of claim 4 or 5, wherein the microparticle has a diameter from about 0.1 micrometers to about 10 micrometers.

7. The method of any one of claims 1-6, wherein the particle is coated with a functional group.

8. The method of claim 7, wherein the functional group is selected from the group consisting of a chemical group, a polynucleotide, a polypeptide, an antibody, a small molecule compound, a peptide and a carbohydrate.

9. The method of claim 7, wherein the chemical group is aldehyde, hydroxyl, carboxyl, ester, amine, sulfo, or sulfhydryl; and/or wherein the polypeptide is streptavidin, neutravidin, or avidin; and/or wherein the polynucleotide is poly-dT or poly-dA.

10. The method of any one of claims 1-9, wherein the target molecule is modified.

11. The method of claim 10, wherein the modification of the target molecule is selected from the group consisting of a chemical group, a polynucleotide, a polypeptide, an antibody, a small molecule compound, a peptide and a carbohydrate.

12. The method of any one of claims 10-11, wherein the target molecule is coupled to the particle through an interaction between the modification of the target molecule and the functional group on the particle.

13. The method of any one of claims 1-12, wherein each particle is coupled with at least one target molecule.

14. The method of any one of claims 1-13, wherein the target molecule is associated with a disease caused by an infectious or pathogenic agent selected from the group consisting of a fungus, a bacterium, a mycoplasma, a rickettsia, a chlamydia, a virus and a protozoa.

15. The method of any one of claims 1-14, wherein the target molecule is associated with a sexually transmitted disease, cancer, cerebrovascular disease, heart disease, respiratory disease, coronary heart disease, diabetes, hypertension, Alzheimer's disease, neurodegenerative disease, chronic obstructive pulmonary disease, autoimmune disease, cystic fibrosis, spinal muscular atrophy, beta thalassemia, phenylalanine hydroxylase deficiency, Duchenne muscular dystrophy, or hereditary hearing loss.

16. The method of any one of claims 1-15, wherein the probe molecule comprises a polynucleotide, a polypeptide, an antibody, a small molecule compound, a peptide and a carbohydrate.

17. The method of any one of claims 1-16, wherein the microarray comprises at least one Tag sequence comprising a polynucleotide sequence selected from the group consisting of SEQ ID NOs: 1-18.

18. The method of any one of claims 1-17, wherein the microarray comprises at least one Tag sequence comprising a polynucleotide sequence selected from the group consisting of SEQ ID NOs: 19-23.

19. The method of any one of claims 1-18, wherein the microarray comprises at least one Tag sequence as set forth in Table 1.

20. The method of any one of claims 1-19, wherein the microarray comprises at least two probe molecules.

21. The method of claim 20, wherein the at least two probe molecules comprise a probe molecule comprising the polynucleotide sequence set forth in SEQ ID NO: 1, and a probe molecule comprising the polynucleotide sequence set forth in SEQ ID NO: 2.

22. The method of claim 20 or 21, wherein the at least two probe molecules comprise a probe molecule comprising the polynucleotide sequence set forth in SEQ ID NO: 3, and a probe molecule comprising the polynucleotide sequence set forth in SEQ ID NO: 4.

23. The method of any one of claims 20-22, wherein the at least two probe molecules comprise a probe molecule comprising the polynucleotide sequence set forth in SEQ ID NO: 5, and a probe molecule comprising the polynucleotide sequence set forth in SEQ ID NO: 6.

24. The method of any one of claims 20-23, wherein the at least two probe molecules comprise a probe molecule comprising the polynucleotide sequence set forth in SEQ ID NO: 7, and a probe molecule comprising the polynucleotide sequence set forth in SEQ ID NO: 8.

25. The method of any one of claims 20-24, wherein the at least two probe molecules comprise a probe molecule comprising the polynucleotide sequence set forth in SEQ ID NO: 9, and a probe molecule comprising the polynucleotide sequence set forth in SEQ ID NO: 10.

26. The method of any one of claims 20-25, wherein the at least two probe molecules comprise a probe molecule comprising the polynucleotide sequence set forth in SEQ ID NO: 11, and a probe molecule comprising the polynucleotide sequence set forth in SEQ ID NO: 12.

27. The method of any one of claims 20-26, wherein the at least two probe molecules comprise a probe molecule comprising the polynucleotide sequence set forth in SEQ ID NO: 13, and a probe molecule comprising the polynucleotide sequence set forth in SEQ ID NO: 14.

28. The method of any one of claims 20-27, wherein the at least two probe molecules comprise a probe molecule comprising the polynucleotide sequence set forth in SEQ ID NO: 15, and a probe molecule comprising the polynucleotide sequence set forth in SEQ ID NO: 16.

29. The method of any one of claims 20-28, wherein the at least two probe molecules comprise a probe molecule comprising the polynucleotide sequence set forth in SEQ ID NO: 17, and a probe molecule comprising the polynucleotide sequence set forth in SEQ ID NO: 18.

30. The method of any one of claims 1-29, wherein the microarray is fabricated using a technology selected from the group consisting of printing with a fine-pointed pin, photolithography using a pre-made mask, photolithography using a dynamic micromirror device, ink-jet printing, microcontact printing, and electrochemistry on a microelectrode array.

31. The method of any one of claims 1-30, wherein the supporting material of the microarray is selected from the group consisting of silicon, glass, plastic, hydrogel, agarose, nitrocellulose and nylon.

32. The method of any one of claims 1-31, wherein a spot on the microarray ranges from about 10 micrometers to about 5000 micrometers in diameter.

33. The method of any one of claims 1-32, wherein the probe molecule is attached to the microarray by in situ synthesis, nonspecific adsorption, specific binding, nonspecific chemical ligation, or chemoselective ligation.

34. The method of any one of claims 1-33, wherein the binding between the target molecule and the probe molecule is a non-covalent, reversible covalent or irreversible covalent interaction.

35. The method of claim 34, wherein the efficiency and/or efficacy of the interaction is enhanced by an external force.

36. The method of claim 35, wherein the external force is a magnetic force, a dielectrophoretic force, a mechanical force, or a combination thereof.

37. The method of any one of claims 1-36, wherein the target molecule is subject to an in vitro manipulation.

38. The method of claim 37, wherein the in vitro manipulation is selected from the group consisting of physical treatments including laser, ultrasonication, heat, microwave, piezoelectricity, electrophoresis, dielectrophoresis, solid phase adhesion, filtration and fluidic stress, and other treatments including enzymatic digestion, PCR amplification, reverse-transcription, reverse-transcription PCR amplification, allele-specific PCR (ASPCR), single-base extension (SBE), allele specific primer extension (ASPE), restriction enzyme digestion, strand displacement amplification (SDA), transcription mediated amplification (TMA), ligase chain reaction (LCR), nucleic acid sequence based amplification (NASBA), primer extension, rolling circle amplification (RCA), self sustained sequence replication (3 SR), the use of Q Beta replicase, nick translation, and loop-mediated isothermal amplification (LAMP).

39. The method of claim 37 or 38, wherein the target molecule comprises a double-stranded polynucleotide, and wherein the double-stranded polynucleotide is denatured to become single-stranded by a chemical reaction, an enzyme, heating, or a combination thereof.

40. The method of claim 39, wherein the enzyme is an exonuclease, a Uracil-N-glycosylase, or a combination thereof.

41. The method of claim 39, wherein the chemical reaction uses urea, formamide, methanol, ethanol, sodium hydroxide, or a combination thereof.

42. The method of claim 39, wherein the double-stranded polynucleotide is denatured at an appropriate temperature from about 30° C. to about 95° C.

43. The method of any one of claims 37-42, wherein the in vitro manipulation is allele-specific PCR (ASPCR).

44. The method of claim 43, wherein the set of primers for the ASPCR comprises at least two allele-specific primers and one common primer.

45. The method of claim 44, wherein the at least two allele-specific primers comprise:
(a) a primer comprising the polynucleotide sequence set forth in SEQ ID NO: 24, and a primer comprising the polynucleotide sequence set forth in SEQ ID NO: 25; and/or
(b) a primer comprising the polynucleotide sequence set forth in SEQ ID NO: 27, and a primer comprising the polynucleotide sequence set forth in SEQ ID NO: 28; and/or
(c) a primer comprising the polynucleotide sequence set forth in SEQ ID NO: 30, and a primer comprising the polynucleotide sequence set forth in SEQ ID NO: 31; and/or
(d) a primer comprising the polynucleotide sequence set forth in SEQ ID NO: 33, and a primer comprising the polynucleotide sequence set forth in SEQ ID NO: 34; and/or
(e) a primer comprising the polynucleotide sequence set forth in SEQ ID NO: 36, and a primer comprising the polynucleotide sequence set forth in SEQ ID NO: 37; and/or
(f) a primer comprising the polynucleotide sequence set forth in SEQ ID NO: 39, and a primer comprising the polynucleotide sequence set forth in SEQ ID NO: 40; and/or
(g) a primer comprising the polynucleotide sequence set forth in SEQ ID NO: 42, and a primer comprising the polynucleotide sequence set forth in SEQ ID NO: 43; and/or
(h) a primer comprising the polynucleotide sequence set forth in SEQ ID NO: 45, and a primer comprising the polynucleotide sequence set forth in SEQ ID NO: 46; and/or
(i) a primer comprising the polynucleotide sequence set forth in SEQ ID NO: 48, and a primer comprising the polynucleotide sequence set forth in SEQ ID NO: 49; and/or
(j) a pair of allele-specific primers as set forth in Table 2.

46. The method of claim 44 or 45, wherein the common primer comprises:
(a) a primer comprising the polynucleotide sequence set forth in SEQ ID NO: 26; and/or
(b) a primer comprising the polynucleotide sequence set forth in SEQ ID NO: 29; and/or
(c) a primer comprising the polynucleotide sequence set forth in SEQ ID NO: 32; and/or
(d) a primer comprising the polynucleotide sequence set forth in SEQ ID NO: 35; and/or
(e) a primer comprising the polynucleotide sequence set forth in SEQ ID NO: 38; and/or
(f) a primer comprising the polynucleotide sequence set forth in SEQ ID NO: 41; and/or
(g) a primer comprising the polynucleotide sequence set forth in SEQ ID NO: 44; and/or
(h) a primer comprising the polynucleotide sequence set forth in SEQ ID NO: 47; and/or
(i) a primer comprising the polynucleotide sequence set forth in SEQ ID NO: 50; and/or
(j) a common primer as set forth in Table 2.

47. The method of any one of claims 1-46, wherein the target molecule is modified by a biotin, a digoxin, or a combination thereof.

48. The method of any one of claims 1-46, wherein the target polynucleotide is modified by a poly-dA or poly-dT.

49. The method of claims 37-48, wherein the target molecule is coupled to the particle through a streptavidin/biotin interaction, a neutravidin/biotin interaction, an avidin/biotin interaction, or a poly-dT/dA interaction.

50. The method of any one of claims 1-49, wherein the luminophore is selected from the group consisting of a fluorophores, a phosphor, and a chromophore.

51. The method of claim 50, wherein the fluorophore is a quantum dot, a protein (e.g., green fluorescent protein) or a small molecule dye.

52. The method of claim 51, wherein the small molecule dye includes: xanthene derivatives (fluorescein, rhodamine, Oregon green, eosin, texas red, etc.), cyanine derivatives (cyanine, indocarbocyanine, oxacarbocyanine, thiacarbocyanine, merocyanine, etc.), naphthalene derivatives (dansyl and prodan derivatives), coumarin derivatives, oxadiazole derivatives (pyridyloxazole, nitrobenzoxadiazole, benzoxadiazole, etc.), pyrene derivatives (cascade blue, etc.), BODIPY (Invitrogen), oxazine derivatives (Nile red, Nile blue, cresyl violet, oxazine 170, etc.), acridine derivatives (proflavin, acridine orange, acridine yellow, etc.), arylmethine derivatives (auramine, crystal violet, malachite green, etc.), CF dye (Biotium), Alexa Fluor (Invitrogen), Atto and Tracy (Sigma), Tetrapyrrole derivatives (porphin, phtalocyanine, bilirubin, etc.), and others (cascade yellow, azure B, acridine orange, DAPI, Hoechst 33258, lucifer yellow, piroxicam, quinine and anthraqinone, squarylium, oligophenylenes, etc.).

53. The method of claim 50, wherein the chromophore includes retinal (used in the eye to detect light), various food colorings, fabric dyes (azo compounds), lycopene, β-carotene, anthocyanins, chlorophyll, hemoglobin, hemocyanin, and colorful minerals such as malachite and amethyst.

54. The method of any one of claims 1-53, wherein the target molecule is directly labeled with a luminophore, or indirectly through the modification of the target molecule, which is selected from the group consisting of a chemical group, a polynucleotide, a polypeptide, an antibody, a small molecule compound, a peptide, and a carbohydrate.

55. The method of any one of claims 1-54, wherein the target molecule is labeled with a luminophore directly, during the in vitro manipulation, or after the in vitro manipulation.

56. The method of any one of claims 1-55, wherein the probe molecule is a polynucleotide.

57. The method of any one of claims 1-56, wherein the target molecule comprises a universal Tag sequence.

58. The method of claim 57, wherein the Tm difference between different Tag sequences equals or is less than about 5° C.

59. The method of claim 57 or 58, wherein the Tag sequences have no cross-hybridization among themselves.

60. The method of any one of claims 57-59, wherein the Tag sequences have low homology to the genomic DNA of the species.

61. The method of any one of claims 57-60, wherein the Tag sequences have no hair-pin structures.

62. The method of any one of claims 57-61, wherein the Tag sequence is a single stranded oligonucleotide or modified analog.

63. The method of any one of claims 57-62, wherein the Tag sequence is a locked nucleic acid (LNA), a Zip nucleic acid (ZNA), or a peptide nucleic acid (PNA).

64. The method of any one of claims 57-63, wherein the Tag sequence is introduced to the target polynucleotide during an in vitro manipulation.

65. The method of any one of claims 1-64, wherein the detection is by a microarray scanning device, an ordinary image-capturing device, or a naked eye.

66. The method of claim 65, wherein the microarray scanning device employs optical detection with a fluorescent label, a chemiluminescent label, a phosphore label, or a chromophore label.

67. The method of claim 65, wherein the microarray scanning device employs label-free detection based on surface plasmon resonance, magnetic force, giant magnetoresistance or microgravimetric technique.

68. The method of claim 65, wherein the ordinary image-capturing device is a flatbed scanner, a camera, or a portable device.

69. The method of claim 68, wherein the camera is with or without the assistance of a lens, a magnifier, or a microscope.

70. The method of claim 68, wherein the portable device is a camera on a mobile phone or a laptop computer with or without the assistance of a lens, a magnifier, or a microscope.

71. A method for detecting a genetic information in a target molecule using a microarray, which method comprises:
(a) labeling a target molecule with a luminophore, wherein the target molecule comprises a polynucleotide comprising a genetic information which comprises:
(1) a genetic information within the target gene of GJB6 (Cx30); and/or
(2) c.132G>C and/or c.269T>C within the target gene of GJB2; and/or
(3) c.1246A>C within the target gene of SLC26A4; and/or
(4) m.7444G>A within the target gene of 12S rRNA;
(b) coupling the target molecule to a particle;
(c) binding the target molecule to a probe molecule immobilized on the microarray; and
(d) detecting the interaction between the target molecule and the probe molecule, thereby detecting the genetic information in the target molecule.

72. The method of claim 71, wherein the genetic information within the target gene of GJB6 (Cx30) is c.del309kb.

73. The method of claim 71 or 72, wherein the polynucleotide further comprises one or more of the following genetic information: c.35delG within the target gene of GJB2; c.167delT within the target gene of GJB2; c.707T>C within the target gene of SLC26A4; and m.1555A>G within the target gene of 12S rRNA.

74. The method of any one of claims 71-73, wherein the genetic information is a mutation selected from the group consisting of a substitution, an insertion, a deletion and an indel.

75. The method of any one of claims 71-73, wherein the genetic information is a single nucleotide polymorphism (SNP).

76. The method of any one of claims 71-75, wherein the genetic information is associated with a disease caused by an infectious or pathogenic agent selected from the group consisting of a fungus, a bacterium, a mycoplasma, a rickettsia, a chlamydia, a virus and a protozoa.

77. The method of any one of claims 71-76, wherein the genetic information is associated with a sexually transmitted disease, cancer, cerebrovascular disease, heart disease, respiratory disease, coronary heart disease, diabetes, hypertension, Alzheimer's disease, neurodegenerative disease, chronic obstructive pulmonary disease, autoimmune disease, cystic fibrosis, spinal muscular atrophy, beta thalassemia, phenylalanine hydroxylase deficiency, Duchenne muscular dystrophy, or hereditary hearing loss.

78. The method of claim 77, wherein the genetic information is associated with hereditary hearing loss.

79. The method of any one of claims 71-78, wherein ASPCR is used to amplify the genetic information.

80. The method of claim 79, wherein the set of primers for the ASPCR includes at least two allele-specific primers and one common primer.

81. The method of claim 80, wherein the at least two allele-specific primers comprise:
(a) a primer comprising the polynucleotide sequence set forth in SEQ ID NO: 24, and a primer comprising the polynucleotide sequence set forth in SEQ ID NO: 25; and/or
(b) a primer comprising the polynucleotide sequence set forth in SEQ ID NO: 27, and a primer comprising the polynucleotide sequence set forth in SEQ ID NO: 28; and/or
(c) a primer comprising the polynucleotide sequence set forth in SEQ ID NO: 30, and a primer comprising the polynucleotide sequence set forth in SEQ ID NO: 31; and/or
(d) a primer comprising the polynucleotide sequence set forth in SEQ ID NO: 33, and a primer comprising the polynucleotide sequence set forth in SEQ ID NO: 34; and/or
(e) a primer comprising the polynucleotide sequence set forth in SEQ ID NO: 36, and a primer comprising the polynucleotide sequence set forth in SEQ ID NO: 37; and/or
(f) a primer comprising the polynucleotide sequence set forth in SEQ ID NO: 39, and a primer comprising the polynucleotide sequence set forth in SEQ ID NO: 40; and/or
(g) a primer comprising the polynucleotide sequence set forth in SEQ ID NO: 42, and a primer comprising the polynucleotide sequence set forth in SEQ ID NO: 43; and/or
(h) a primer comprising the polynucleotide sequence set forth in SEQ ID NO: 45, and a primer comprising the polynucleotide sequence set forth in SEQ ID NO: 46; and/or
(i) a primer comprising the polynucleotide sequence set forth in SEQ ID NO: 48, and a primer comprising the polynucleotide sequence set forth in SEQ ID NO: 49; and/or
(j) a pair of allele-specific primers as set forth in Table 2.

82. The method of claim 80 or 81, wherein the common primer comprises:
(a) a primer comprising the polynucleotide sequence set forth in SEQ ID NO: 26; and/or
(b) a primer comprising the polynucleotide sequence set forth in SEQ ID NO: 29; and/or
(c) a primer comprising the polynucleotide sequence set forth in SEQ ID NO: 32; and/or
(d) a primer comprising the polynucleotide sequence set forth in SEQ ID NO: 35; and/or
(e) a primer comprising the polynucleotide sequence set forth in SEQ ID NO: 38; and/or
(f) a primer comprising the polynucleotide sequence set forth in SEQ ID NO: 41; and/or
(g) a primer comprising the polynucleotide sequence set forth in SEQ ID NO: 44; and/or
(h) a primer comprising the polynucleotide sequence set forth in SEQ ID NO: 47; and/or
(i) a primer comprising the polynucleotide sequence set forth in SEQ ID NO: 50; and/or
(j) a common primer as set forth in Table 2.

83. The method of any one of claims 80-82, wherein the common primer is labeled with a luminophore as well as with biotinylation.

84. The method of any one of claims 80-83, wherein the allele-specific primers terminate at the SNP/mutation locus.

85. The method of any one of claims 80-84, wherein the allele-specific primer further comprises an artificial mismatch to the corresponding target sequence.

86. The method of any one of claims 80-85, wherein the allele-specific primers comprise a natural nucleotide or analog thereof.

87. The method of any one of claims 80-86, wherein the allele-specific primers comprise a Tag sequence.

88. The method of any one of claims 79-87, wherein the ASPCR uses a DNA polymerase without the 3′ to 5′ exonuclease activity.

89. The method of any one of claims 71-88, wherein at least two genetic information are detected.

90. The method of any one of claims 71-89, wherein multiplex PCR is used to amplify the genetic information.

91. The method of any one of claims 71-90, wherein the genetic information is detected in a sample selected from the group consisting of tissue, cell, body fluid, hair, nail, ejaculate, saliva, sputum, sperm, oocyte, zygote, lymph, blood, interstitial fluid, urine, buccal swab, chewing gum, cigarette butt, envelope, stamp, a prenatal sample, or dried blood spot.

92. The method of any one of claims 71-91, wherein the microarray comprises at least one Tag sequence comprising a polynucleotide sequence selected from the group consisting of SEQ ID NOs: 1-18.

93. The method of any one of claims 71-92, wherein the microarray comprises at least one Tag sequence comprising a polynucleotide sequence selected from the group consisting of SEQ ID NOs: 19-23.

94. The method of any one of claims 71-93, wherein the microarray comprises at least one Tag sequence as set forth in Table 1.

95. The method of any one of claims 71-94, wherein the microarray comprises at least two probe molecules.

96. The method of claim 95, wherein the at least two probe molecules comprise a probe molecule comprising the polynucleotide sequence set forth in SEQ ID NO: 1, and a probe molecule comprising the polynucleotide sequence set forth in SEQ ID NO: 2.

97. The method of claim 95 or 96, wherein the at least two probe molecules comprise a probe molecule comprising the polynucleotide sequence set forth in SEQ ID NO: 3, and a probe molecule comprising the polynucleotide sequence set forth in SEQ ID NO: 4.

98. The method of any one of claims 95-97, wherein the at least two probe molecules comprise a probe molecule comprising the polynucleotide sequence set forth in SEQ ID NO: 5, and a probe molecule comprising the polynucleotide sequence set forth in SEQ ID NO: 6.

99. The method of any one of claims 95-98, wherein the at least two probe molecules comprise a probe molecule comprising the polynucleotide sequence set forth in SEQ ID NO: 7, and a probe molecule comprising the polynucleotide sequence set forth in SEQ ID NO: 8.

100. The method of any one of claims 95-99, wherein the at least two probe molecules comprise a probe molecule comprising the polynucleotide sequence set forth in SEQ ID NO: 9, and a probe molecule comprising the polynucleotide sequence set forth in SEQ ID NO: 10.

101. The method of any one of claims 95-100, wherein the at least two probe molecules comprise a probe molecule comprising the polynucleotide sequence set forth in SEQ ID NO: 11, and a probe molecule comprising the polynucleotide sequence set forth in SEQ ID NO: 12.

102. The method of any one of claims 95-101, wherein the at least two probe molecules comprise a probe molecule comprising the polynucleotide sequence set forth in SEQ ID NO: 13, and a probe molecule comprising the polynucleotide sequence set forth in SEQ ID NO: 14.

103. The method of any one of claims 95-102, wherein the at least two probe molecules comprise a probe molecule comprising the polynucleotide sequence set forth in SEQ ID NO: 15, and a probe molecule comprising the polynucleotide sequence set forth in SEQ ID NO: 16.

104. The method of any one of claims 95-103, wherein the at least two probe molecules comprise a probe molecule comprising the polynucleotide sequence set forth in SEQ ID NO: 17, and a probe molecule comprising the polynucleotide sequence set forth in SEQ ID NO: 18.

105. A composition comprising a luminophore-labeled target molecule coupled to a particle and a probe molecule immobilized on a microarray that binds to the target molecule, wherein the target molecule comprises a polynucleotide comprising a genetic information which comprises:
(1) a genetic information within the target gene of GJB6 (Cx30); and/or
(2) c.132G>C and/or c.269T>C within the target gene of GJB2; and/or
(3) c.1246A>C within the target gene of SLC26A4; and/or
(4) m.7444G>A within the target gene of 12S rRNA.

106. The composition of claim 105, wherein the genetic information within the target gene of GJB6 (Cx30) is c.del309kb.

107. The composition of claim 105 or 106, wherein the polynucleotide further comprises one or more of the following genetic information: c.35delG within the target gene of GJB2; c.167delT within the target gene of GJB2; c.707T>C within the target gene of SLC26A4; and m.1555A>G within the target gene of 12S rRNA.

108. The composition of any one of claims 105-107, wherein the microarray comprises at least one Tag sequence comprising a polynucleotide sequence selected from the group consisting of SEQ ID NOs: 1-18.

109. The composition of any one of claims 105-108, wherein the microarray comprises at least one Tag sequence comprising a polynucleotide sequence selected from the group consisting of SEQ ID NOs: 19-23.

110. The composition of any one of claims 105-109, wherein the microarray comprises at least one Tag sequence as set forth in Table 1.

111. The composition of any one of claims 105-110, wherein the probe molecule comprises a polynucleotide, a polypeptide, an antibody, a small molecule compound, a peptide and a carbohydrate.

112. The composition of any one of claims 105-111, wherein the particle is a microparticle.

113. The composition of claim 112, wherein the microparticle is a paramagnetic microsphere.

114. The composition of claim 112 or 113, wherein the microparticle has a diameter from about 0.1 micrometers to about 10 micrometers.

115. An oligonucleotide probe comprising a Tag sequence as set forth in Table 1 or a polynucleotide sequence selected from the group consisting of SEQ ID NOs: 1-18.

116. A universal Tag array comprising at least two of the Tag sequences as set forth in Table 1.

117. The universal Tag array of claim 116, comprising all of the Tag sequences set forth in Table 1.

118. A primer comprising a sequence as set forth in Table 2 without the Tag sequence or biotinylated universal primer sequence at the 5′-terminus, which primer is not a full-length cDNA or a full-length genomic DNA.

119. The primer of claim 118, which consists essentially of the sequence as set forth in Table 2 without the Tag sequence or biotinylated universal primer sequence at the 5′-terminus.

120. The primer of claim 118, which consists of the sequence as set forth in Table 2 without the Tag sequence or biotinylated universal primer sequence at the 5′-terminus.

121. The primer of claim 118, which comprises a sequence as set forth in Table 2.

122. A set of primers for ASPCR amplification of a genetic information comprising two allele-specific primers and a luminophore-labeled common primer as set forth in Table 2, wherein the luminophore is TAMRA.

123. A kit useful for detecting a genetic information comprising the universal Tag array of claim 116 or claim 117.

124. The kit of claim 123, further comprising an instructional manual.

125. The kit of claim 123 or 124, further comprising the primer of claims 118-121.

126. The kit of any one of claims 123-125, further comprising the set of primers for ASPCR amplification of a genetic information of claim 122.

127. A kit useful for detecting a molecular interaction comprising a particle, a microarray and at least one probe molecule immobilized on the microarray, wherein the at least one probe molecule comprises a polynucleotide sequence selected from the group consisting of SEQ ID NOs: 1-18.

128. The kit of claim 127, wherein the at least one probe molecule further comprises a polynucleotide sequence selected from the group consisting of SEQ ID NOs: 19-23.

129. The kit of claim 127 or 128, wherein the at least one probe molecule comprises at least one Tag sequence as set forth in Table 1.

130. The kit of any one of claims 127-129, wherein the particle is a microparticle.

131. The kit of claim 130, wherein the microparticle is a paramagnetic microsphere.

132. The kit of claim 130 or 131, wherein the microparticle has a diameter from about 0.1 micrometers to about 10 micrometers.

133. The kit of any one of claims 127-132, wherein the particle is coated with a functional group.

134. The kit of claim 133, wherein the functional group is selected from the group consisting of a chemical group, a polynucleotide, a polypeptide, an antibody, a small molecule compound, a peptide and a carbohydrate.

135. The kit of claim 134, wherein the chemical group is aldehyde, hydroxyl, carboxyl, ester, amine, sulfo, or sulfhydryl.

136. The kit of claim 134, wherein the polypeptide is streptavidin, neutravidin, or avidin.

137. The kit of claim 134, wherein the polynucleotide is poly-dT or poly-dA.


SEQUENCE LISTING (50 sequences)

SEQ ID NO: 1 - DNA, 22 bp, Artificial Sequence
  Primer
gaggagatcgtagctggtgcat

SEQ ID NO: 2 - DNA, 22 bp, Artificial Sequence
  Primer
tcgctgccaaccgagaattgca

SEQ ID NO: 3 - DNA, 24 bp, Artificial Sequence
  Primer
caaggcacgtcccagacgcatcaa

SEQ ID NO: 4 - DNA, 20 bp, Artificial Sequence
  Primer
gaccgaggatagcgttaccg

SEQ ID NO: 5 - DNA, 20 bp, Artificial Sequence
  Primer
ctaacggagagacgcaacgg

SEQ ID NO: 6 - DNA, 24 bp, Artificial Sequence
  Primer
ggtatcgcgaccgcatcccaatct

SEQ ID NO: 7 - DNA, 20 bp, Artificial Sequence
  Primer
ggacggtaatctcgcttcgc

SEQ ID NO: 8 - DNA, 23 bp, Artificial Sequence
  Primer
gttagggtcgcgccaaactctcc

SEQ ID NO: 9 - DNA, 22 bp, Artificial Sequence
  Primer
tgcacgagttgggtgagtttgg

SEQ ID NO: 10 - DNA, 22 bp, Artificial Sequence
  Primer
tcgagtacaggacccacatcag

SEQ ID NO: 11 - DNA, 22 bp, Artificial Sequence
  Primer
ctgttaaacgtcagagcgcagc

SEQ ID NO: 12 - DNA, 22 bp, Artificial Sequence
  Primer
agtcgaagtgtgcgtcagactc

SEQ ID NO: 13 - DNA, 20 bp, Artificial Sequence
  Primer
cgagacgacttaggacgagg

SEQ ID NO: 14 - DNA, 22 bp, Artificial Sequence
  Primer
atgacgacctgagtgcacacac

SEQ ID NO: 15 - DNA, 22 bp, Artificial Sequence
  Primer
gagcaagcgcaaacgcagtact

SEQ ID NO: 16 - DNA, 22 bp, Artificial Sequence
  Primer
gcatagacgtggctcaactgtc

SEQ ID NO: 17 - DNA, 23 bp, Artificial Sequence
  Primer
acctttcgcttcaccggccgatc

SEQ ID NO: 18 - DNA, 23 bp, Artificial Sequence
  Primer
gctcgaagaggcgctacagatcc

SEQ ID NO: 19 - DNA, 17 bp, Artificial Sequence
  Primer
gcaaccaccaccggagg

SEQ ID NO: 20 - DNA, 21 bp, Artificial Sequence
  Primer
tcctcaacggaagcaagtgat

SEQ ID NO: 21 - DNA, 19 bp, Artificial Sequence
  Primer
gcgcttcagggccctgttc

SEQ ID NO: 22 - DNA, 23 bp, Artificial Sequence
  Primer
gctttatccctaacgtcatcggg

SEQ ID NO: 23 - DNA, 21 bp, Artificial Sequence
  Primer
cagagtgcttggtgccataac

SEQ ID NO: 24 - DNA, 20 bp, Artificial Sequence
  Primer
tgtttgttcacacccccgag

SEQ ID NO: 25 - DNA, 19 bp, Artificial Sequence
  Primer
tgtttgttcacacccgcag

SEQ ID NO: 26 - DNA, 20 bp, Artificial Sequence
  Primer
gcatgcttgcttacccagac

SEQ ID NO: 27 - DNA, 20 bp, Artificial Sequence
  Primer
agtcggcctgctcatcttcc

SEQ ID NO: 28 - DNA, 18 bp, Artificial Sequence
  Primer
accctgcagccagctacg

SEQ ID NO: 29 - DNA, 20 bp, Artificial Sequence
  Primer
gcatgcttgcttacccagac

SEQ ID NO: 30 - DNA, 22 bp, Artificial Sequence
  Primer
gactttgtctgcaacacactgc

SEQ ID NO: 31 - DNA, 21 bp, Artificial Sequence
  Primer
gactttgtctgcaacaaccgc

SEQ ID NO: 32 - DNA, 25 bp, Artificial Sequence
  Primer
tccctctcatgctgtctatttctta

SEQ ID NO: 33 - DNA, 17 bp, Artificial Sequence
  Primer
tccacgccagcgcacct

SEQ ID NO: 34 - DNA, 18 bp, Artificial Sequence
  Primer
gtccacgccagcgctctc

SEQ ID NO: 35 - DNA, 25 bp, Artificial Sequence
  Primer
tccctctcatgctgtctatttctta

SEQ ID NO: 36 - DNA, 22 bp, Artificial Sequence
  Primer
cctactacaggcacgaaaccac

SEQ ID NO: 37 - DNA, 25 bp, Artificial Sequence
  Primer
caccatgcgtagccttaaccatttt

SEQ ID NO: 38 - DNA, 22 bp, Artificial Sequence
  Primer
gcacaactctgccacgttaagc

SEQ ID NO: 39 - DNA, 25 bp, Artificial Sequence
  Primer
cttgaaacattgaggacaatctata

SEQ ID NO: 40 - DNA, 25 bp, Artificial Sequence
  Primer
gttgaaacattgaggacaatctgtg

SEQ ID NO: 41 - DNA, 25 bp, Artificial Sequence
  Primer
cagagagtaggtttctatctcaggc

SEQ ID NO: 42 - DNA, 25 bp, Artificial Sequence
  Primer
ttcctacctgtgtctttcctcaagt

SEQ ID NO: 43 - DNA, 24 bp, Artificial Sequence
  Primer
tcctacctgtgtctttcctcctgg

SEQ ID NO: 44 - DNA, 25 bp, Artificial Sequence
  Primer
cgttgtcatccagtctcttccttag

SEQ ID NO: 45 - DNA, 24 bp, Artificial Sequence
  Primer
agaacccgtatacataaaatcgag

SEQ ID NO: 46 - DNA, 24 bp, Artificial Sequence
  Primer
agaacccgtatacataaaatcgaa

SEQ ID NO: 47 - DNA, 21 bp, Artificial Sequence
  Primer
gttaggaaaagggcatacagg

SEQ ID NO: 48 - DNA, 21 bp, Artificial Sequence
  Primer
acttaccatgttacgactagt

SEQ ID NO: 49 - DNA, 22 bp, Artificial Sequence
  Primer
cacttaccatgttacgactcgc

SEQ ID NO: 50 - DNA, 20 bp, Artificial Sequence
  Primer
ccctgatgaaggctacaaag